SEQUENCE LISTING (100 sequences)

SEQ ID NO: 1 - protein, 114 aa, synthetic construct
  REGION 1..114: note = Synthetic: hIL15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 232 aa, synthetic construct
  REGION 1..232: note = Synthetic: hIgG1Fc (Fcdw)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 232 aa, synthetic construct
  REGION 1..232: note = Synthetic: hIgG1Fcmutant (Fcdm):Fcgamma R binding deficiency
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 77 aa, synthetic construct
  REGION 1..77: note = Synthetic: h15Ra (sushi domain)
ITCPPPMSVEHADIWVKSYSLYSRERYICNSGFKRKAGTSSLTECVLNKATNVAHWTTPSLKCIRDPALVHQRPAPP

SEQ ID NO: 5 - protein, 214 aa, synthetic construct
  REGION 1..214: note = Synthetic: h15Rb (27aa˜240aa)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 6 - protein, 208 aa, synthetic construct
  REGION 1..208: note = Synthetic: h15Rb (27aa˜234aa)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPA

SEQ ID NO: 7 - protein, 209 aa, synthetic construct
  REGION 1..209: note = Synthetic: h15Rb (32aa˜240aa)
SQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 8 - protein, 203 aa, synthetic construct
  REGION 1..203: note = Synthetic: h15Rb (32aa˜234aa)
SQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPA

SEQ ID NO: 9 - protein, 99 aa, synthetic construct
  REGION 1..99: note = Synthetic: h15Rb (27aa˜125aa)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFK

SEQ ID NO: 10 - protein, 102 aa, synthetic construct
  REGION 1..102: note = Synthetic: h15Rb (27aa˜128aa)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFE

SEQ ID NO: 11 - protein, 111 aa, synthetic construct
  REGION 1..111: note = Synthetic: h15Rb (27aa˜137aa)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPIS

SEQ ID NO: 12 - protein, 106 aa, synthetic construct
  REGION 1..106: note = Synthetic: h15Rb (32aa˜137aa)
SQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPIS

SEQ ID NO: 13 - protein, 40 aa, synthetic construct
  REGION 1..40: note = Synthetic: Non-cleavable linker
  REGION 1..40: note = misc_feature - "Gly Gly Gly Ser" repeats at least one time and up to 10 times
GGGSGGGSGGGSGGGSGGGSGGGSGGGSGGGSGGGSGGGS

SEQ ID NO: 14 - protein, 50 aa, synthetic construct
  REGION 1..50: note = Synthetic: Non-cleavable linker
  REGION 1..50: note = misc_feature - "Gly Gly Gly Gly Ser" repeats at least one time and up to 10times
GGGGSGGGGSGGGGSGGGGSGGGGSGGGGSGGGGSGGGGSGGGGSGGGGS

SEQ ID NO: 15 - protein, 50 aa, synthetic construct
  REGION 1..50: note = Synthetic: Non-cleavable linker
  REGION 1..50: note = misc_feature - "Ser Gly Gly Gly Ser" repeats at least one time and up to 10times
SGGGSSGGGSSGGGSSGGGSSGGGSSGGGSSGGGSSGGGSSGGGSSGGGS

SEQ ID NO: 16 - protein, 26 aa, synthetic construct
  REGION 1..26: note = Synthetic: Non-cleavable linker
SGGGSGGGGSGGGGSGGGGSGGGSLQ

SEQ ID NO: 17 - protein, 10 aa, synthetic construct
  REGION 1..10: note = Synthetic: Cleavable mmp substrate
SGRSPAIFTA

SEQ ID NO: 18 - protein, 10 aa, synthetic construct
  REGION 1..10: note = Synthetic: Cleavable mmp substrate
SGRSENIRTA

SEQ ID NO: 19 - protein, 10 aa, synthetic construct
  REGION 1..10: note = Synthetic: Cleavable mmp substrate
SGARYRWLTA

SEQ ID NO: 20 - protein, 10 aa, synthetic construct
  REGION 1..10: note = Synthetic: Cleavable mmp substrate
SGRSYAILTA

SEQ ID NO: 21 - protein, 10 aa, synthetic construct
  REGION 1..10: note = Synthetic: Cleavable mmp substrate
SGRAMHMYTA

SEQ ID NO: 22 - protein, 7 aa, synthetic construct
  REGION 1..7: note = Synthetic: Cleavable mmp substrate
SPVGLIG

SEQ ID NO: 23 - protein, 10 aa, synthetic construct
  REGION 1..10: note = Synthetic: Cleavable mmp substrate
SGRIGFLRTA

SEQ ID NO: 24 - protein, 10 aa, synthetic construct
  REGION 1..10: note = Synthetic: Cleavable mmp substrate
SGAIGFLRTA

SEQ ID NO: 25 - protein, 10 aa, synthetic construct
  REGION 1..10: note = Synthetic: Cleavable mmp substrate
SGAAMHMYTA

SEQ ID NO: 26 - protein, 10 aa, synthetic construct
  REGION 1..10: note = Synthetic: Cleavable mmp substrate
SGASENIRTA

SEQ ID NO: 27 - protein, 10 aa, synthetic construct
  REGION 1..10: note = Synthetic: Cleavable mmp substrate
SGRPENIRTA

SEQ ID NO: 28 - protein, 10 aa, synthetic construct
  REGION 1..10: note = Synthetic: Cleavable mmp substrate
SGAPENIRTA

SEQ ID NO: 29 - protein, 10 aa, synthetic construct
  REGION 1..10: note = Synthetic: Cleavable mmp substrate
SGLISHSITA

SEQ ID NO: 30 - protein, 10 aa, synthetic construct
  REGION 1..10: note = Synthetic: Cleavable mmp substrate
SGNLRSKLTA

SEQ ID NO: 31 - protein, 10 aa, synthetic construct
  REGION 1..10: note = Synthetic: Cleavable mmp substrate
SGVFSIPLTA

SEQ ID NO: 32 - protein, 10 aa, synthetic construct
  REGION 1..10: note = Synthetic: Cleavable mmp substrate
SGIKYHSLTA

SEQ ID NO: 33 - protein, 21 aa, synthetic construct
  REGION 1..21: note = Synthetic: Cleavable MMP substrate sequence-long linker
SGRSPAIFTALSGRSENIRTA

SEQ ID NO: 34 - protein, 21 aa, synthetic construct
  REGION 1..21: note = Synthetic: Cleavable MMP substrate sequence-long linker
SGRSENIRTALSGRSPAIFTA

SEQ ID NO: 35 - protein, 21 aa, synthetic construct
  REGION 1..21: note = Synthetic: Cleavable MMP substrate sequence-long linker
SGARYRWLTALSGRSENIRTA

SEQ ID NO: 36 - protein, 21 aa, synthetic construct
  REGION 1..21: note = Synthetic: Cleavable MMP substrate sequence-long linker
SGRSENIRTALSGARYRWLTA

SEQ ID NO: 37 - protein, 21 aa, synthetic construct
  REGION 1..21: note = Synthetic: Cleavable MMP substrate sequence-long linker
SGRAMHMYTALSGRSENIRTA

SEQ ID NO: 38 - protein, 21 aa, synthetic construct
  REGION 1..21: note = Synthetic: Cleavable MMP substrate sequence-long linker
SGRAMHMYTALSGRAMHMYTA

SEQ ID NO: 39 - protein, 21 aa, synthetic construct
  REGION 1..21: note = Synthetic: Cleavable MMP substrate sequence-long linker
SGRSPAIFTALSGARYRWLTA

SEQ ID NO: 40 - protein, 21 aa, synthetic construct
  REGION 1..21: note = Synthetic: Cleavable MMP substrate sequence-long linker
SGARYRWLTALSGRSPAIFTA

SEQ ID NO: 41 - protein, 21 aa, synthetic construct
  REGION 1..21: note = Synthetic: Cleavable MMP substrate sequence-long linker
SGRSPAIFTALSGRSYAILTA

SEQ ID NO: 42 - protein, 21 aa, synthetic construct
  REGION 1..21: note = Synthetic: Cleavable MMP substrate sequence-long linker
SGRSYAILTALSGRSPAIFTA

SEQ ID NO: 43 - protein, 21 aa, synthetic construct
  REGION 1..21: note = Synthetic: Cleavable MMP substrate sequence-long linker
SGRSPAIFTALSGRAMHMYTA

SEQ ID NO: 44 - protein, 21 aa, synthetic construct
  REGION 1..21: note = Synthetic: Cleavable MMP substrate sequence-long linker
SGRAMHMYTALSGRSPAIFTA

SEQ ID NO: 45 - protein, 21 aa, synthetic construct
  REGION 1..21: note = Synthetic: Cleavable MMP substrate sequence-long linker
SGARYRWLTALSGRSYAILTA

SEQ ID NO: 46 - protein, 21 aa, synthetic construct
  REGION 1..21: note = Synthetic: Cleavable MMP substrate sequence-long linker
SGRSYAILTALSGARYRWLTA

SEQ ID NO: 47 - protein, 21 aa, synthetic construct
  REGION 1..21: note = Synthetic: Cleavable MMP substrate sequence-long linker
SGARYRWLTALSGRAMHMYTA

SEQ ID NO: 48 - protein, 20 aa, synthetic construct
  REGION 1..20: note = Synthetic: Cleavable MMP substrate sequence-long linker
SGRAMHMYTALSGARYRWLT

SEQ ID NO: 49 - protein, 21 aa, synthetic construct
  REGION 1..21: note = Synthetic: Cleavable MMP substrate sequence-long linker
SGRSYAILTALSGRAMHMYTA

SEQ ID NO: 50 - protein, 21 aa, synthetic construct
  REGION 1..21: note = Synthetic: Cleavable MMP substrate sequence-long linker
SGRAMHMYTALSGRSYAILTA

SEQ ID NO: 51 - protein, 103 aa, synthetic construct
  REGION 1..103: note = Synthetic: CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 52 - protein, 107 aa, synthetic construct [sequence omitted]

SEQ ID NO: 53 - protein, 21 aa, synthetic construct
  REGION 1..21: note = Synthetic: IgkL sequence
METDTLLLWVLLLWVPGSTGD

SEQ ID NO: 54 - protein, 305 aa, Homo sapiens
  REGION 1..305: note = misc_feature - human IL-12 P40 subunit (no signal peptide)
WELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 55 - protein, 197 aa, Homo sapiens
  REGION 1..197: note = misc_feature - human IL-12P35 subunit (no signal peptide)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 56 - protein, 227 aa, Homo sapiens
  REGION 1..227: note = misc_feature - mutant human Fc-hole (Fc-h)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 229 aa, Homo sapiens
  REGION 1..229: note = misc_feature - mutant human Fc-knob (Fc-k)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKHV

SEQ ID NO: 58 - protein, 235 aa, Homo sapiens
  REGION 1..235: note = misc_feature - human IL-12R-beta-1
MEPLVTWVVPLLFLFAASRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRRLGSQGSSWSKWSSPVCVPPEN

SEQ ID NO: 59 - protein, 319 aa, Homo sapiens
  REGION 1..319: note = misc_feature - human IL-12R-beta-2
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 60 - protein, 909 aa, synthetic construct
  REGION 1..909: note = Synthetic: 28.1: VH(ahPDL1)LCH1FcL(*)hIFNa2mLFc (1213)
METDTLLLWVLLLWVPGSTGDQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 234 aa, synthetic construct
  REGION 1..234: note = Synthetic: 28.2: VL (ahPDL1)CL (1143)
METDTLLLWVLLLWVPGSTGDEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 910 aa, synthetic construct
  REGION 1..910: note = Synthetic: 29.1: VH(ahPDL1)LCH1FcL hIFNa2mL(*)Fc (1221)
METDTLLLWVLLLWVPGSTGDQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGAGGSGRSGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGARYRWLGRSENIRTGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 234 aa, synthetic construct; identical to SEQ ID NO: 61
  REGION 1..234: note = Synthetic: VL (a-hPDL1)-CL (1143)

SEQ ID NO: 64 - protein, 1168 aa, synthetic construct
  REGION 1..1168: note = Synthetic: 30.1: VH(aPDL1)CH1LFcL (*)hIFNa2mLFcLscFV (aCTLA4)(1222)
METDTLLLWVLLLWVPGSTGDQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRPHSRGGGSGGGSTGRSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 65 - protein, 234 aa, synthetic construct; identical to SEQ ID NO: 61
  REGION 1..234: note = Synthetic: 28.2: VL (ahPDL1)CL(1143)

SEQ ID NO: 66 - protein, 1169 aa, synthetic construct
  REGION 1..1169: note = Synthetic: 31.1: VH(aPDL1) LCH1FcLhIFNa2mL(*)FcL scFV (ahCTLA4)(1264)
METDTLLLWVLLLWVPGSTGDQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGAGGSGRSGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGARYRWLGRSENIRTGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRPHSRGGGSGGGSTGRSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 67 - protein, 1159 aa, synthetic construct
  REGION 1..1159: note = Synthetic: 32.1: VH (ahPDL1)LCH1FcLVH (aCTLA4)L (*)hIFNa2mLFcLVL(aCTLA4)(1269)
METDTLLLWVLLLWVPGSTGDQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGTGGFESQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGARYRWLTAGGSGRSENIRTAGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRPHSRGGGSGGGSTGRSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 68 - protein, 234 aa, synthetic construct; identical to SEQ ID NO: 61
  REGION 1..234: note = Synthetic: 28.2: VL (ahPDL1)CL (1143)

SEQ ID NO: 69 - protein, 714 aa, synthetic construct
  REGION 1..714: note = Synthetic: Ra (sushi)-L-Fc-L (*)-IL15-L-Fc (1156)
METDTLLLWVLLLWVPGSTGDITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 897 aa, synthetic construct
  REGION 1..897: note = Synthetic: Ra (sushi)-L-Fc-L (*)-IL15-L-Fc-L-IFNam2(1224)
METDTLLLWVLLLWVPGSTGDITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRPHSRGGGSGGGSTGRSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKE

SEQ ID NO: 71 - protein, 967 aa, synthetic construct
  REGION 1..967: note = Synthetic: scFV (a-hPD1)-L-Ra (sushi)-L-Fc-L (*)-IL15-L-F2c(1255)
METDTLLLWVLLLWVPGSTGDEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGSGGITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 884 aa, synthetic construct
  REGION 1..884: note = Synthetic: hIFNa2m-L-Ra (sushi)-L-Fc-L (*)-IL15-L-Fc (1256)
METDTLLLWVLLLWVPGSTGDCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGGSITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 960 aa, synthetic construct
  REGION 1..960: note = Synthetic: scFV (a-CTLA4)-L-Ra (sushi)-L-Fc-L (*)-IL15-L-Fc(1257)
METDTLLLWVLLLWVPGSTGDEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 935 aa, synthetic construct
  REGION 1..935: note = Synthetic: 15Rb-L(*)-Ra (sushi)-L-Fc-L(*)-IL15-L-Fc (1198B)
METDTLLLWVLLLWVPGSTGDSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPASENIRTASGRSPAIFTASITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 1118 aa, synthetic construct
  REGION 1..1118: note = Synthetic: 15Rb-L (*)-Ra (sushi)-L-Fc-L(*)-IL15-L-Fc-L-hIFNa2m(1229)
METDTLLLWVLLLWVPGSTGDSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPASENIRTASGRSPAIFTASITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRPHSRGGGSGGGSTGRSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKE

SEQ ID NO: 76 - protein, 954 aa, synthetic construct
  REGION 1..954: note = Synthetic: Ra (sushi)-L-Fc-L(*)-IL15-L-Fc-L(*)-15Rb (1199C)
METDTLLLWVLLLWVPGSTGDITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTGGSGRSPAIFTAGGGSRSENIRTSGGTGGSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTG

SEQ ID NO: 77 - protein, 1207 aa, synthetic construct
  REGION 1..1207: note = Synthetic: scFV (a-hPD1)-L- Ra(sushi)-L-Fc-L(*)-IL15-L-Fc-L(*)-15Rb (1225)
METDTLLLWVLLLWVPGSTGDEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGSGGITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTGGSGRSPAIFTAGGGSRSENIRTSGGTGGSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTG

SEQ ID NO: 78 - protein, 1124 aa, synthetic construct
  REGION 1..1124: note = Synthetic: hIFNam2-L- Ra (sushi)-L-Fc-L(*)-IL15-L-Fc-L(*)-15Rb(1226)
METDTLLLWVLLLWVPGSTGDCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFRRITLYLKEKKYSPCAWEVVRAEIMRSFSLSTNLQESLRSKESGGGSITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTGGSGRSPAIFTAGGGSRSENIRTSGGTGGSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTG

SEQ ID NO: 79 - protein, 1200 aa, synthetic construct
  REGION 1..1200: note = Synthetic: scFV (a-hCTLA4)-L- Ra(sushi)-L-Fc-L(*)-IL15-L-Fc-L(*)-15Rb (1232)
METDTLLLWVLLLWVPGSTGDEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGITCPPPMSVEHADIWVKSYSLYSRERYICNSGFKRKAGTSSLTECVLNKATNVAHWTTPSLKCIRDPALVHQRPAPPGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSGARYRWLTAGGSGRSENIRTAGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTGGSGRSPAIFTAGGGSRSENIRTSGGTGGSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTG

SEQ ID NO: 80 - protein, 1016 aa, synthetic construct
  REGION 1..1016: note = Synthetic: hIL12P40-L-Fc-L(*)-hIL12P35-L-Fc (IT25)
METDTLLLWVLLLWVPGSTGDIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGSGRSENIRTATGGGGSGGGSGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 1255 aa, synthetic construct
  REGION 1..1255: note = Synthetic: 12Rb1-L(*)-hIL12P40-L-Fc-L(*)-hIL12P35-L-Fc(IT30)
METDTLLLWVLLLWVPGSTGDCRTECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENLEGGGGSGRSENIRTATGGGGSGGGSGTIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGSGRSENIRTATGGGGSGGGSGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 1347 aa, synthetic construct
  REGION 1..1347: note = Synthetic: hIL12P40-L-Fc- L2(*)-hIL122P35-L-Fc-L (*)-12Rb2 (IT32)
METDTLLLWVLLLWVPGSTGDIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGSGRSENIRTATGGGGSGGGSGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSSGARYRWLTAGGSGRAMHMYTAGGRSGGIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 83 - protein, 1586 aa, synthetic construct
  REGION 1..1586: note = Synthetic: 12Rb1-L (*)-hIL12P40-L-Fc-L (*)-hIL12P35-L-Fc-L(*)-12Rb2 (IT33)
METDTLLLWVLLLWVPGSTGDCRTECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENLEGGGGSGRSENIRTATGGGGSGGGSGTIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGSGRSENIRTATGGGGSGGGSGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSSGARYRWLTAGGSGRAMHMYTAGGRSGGIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 84 - protein, 1605 aa, synthetic construct
  REGION 1..1605: note = Synthetic: VL-L-VH (a-hPD1)-L-hIL12P40-L-Fc-L(*)-hI L12P35-L-Fc-L(*)-12Rb2(IT43)
METDTLLLWVLLLWVPGSTGDEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGSRFESGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGSGRSENIRTATGGGGSGGGSGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSSGARYRWLTAGGSGRAMHMYTAGGRSGGIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 85 - protein, 1278 aa, synthetic construct
  REGION 1..1278: note = Synthetic: VL-L-VH (a-hPD1)-L-IL-12hP40-L-Fc-L(*)-hIL12P35-L-Fc(IT46)
METDTLLLWVLLLWVPGSTGDEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGSRFESGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEGGGGSGRSENIRTATGGGGSGGGSGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 1292 aa, synthetic construct
  REGION 1..1292: note = Synthetic: VL-L-VH (a-hPD1)-L-Fc-L-hIL-12P40-L(*)-Fc-L-hIL12P35(IT49)
METDTLLLWVLLLWVPGSTGDEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSRFESGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSSGARYRWLTAGGSGRSENIRTGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRPHSRGGGSGGGSTGRSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 87 - protein, 1619 aa, synthetic construct
  REGION 1..1619: note = Synthetic: VL-L-VH(a-hPD1)-Fc-L-hIL-12P40-L(*)-Fc-L-hP35-L(*)-12Rb2 (IT50)
METDTLLLWVLLLWVPGSTGDEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSRFESGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSSGARYRWLTAGGSGRSENIRTGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSRPHSRGGGSGGGSTGRSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSSGARYRWLTAGGSGRAMHMYTAGGRSGGIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 88 - protein, 133 aa, Homo sapiens [sequence omitted]

SEQ ID NO: 89 - protein, 152 aa, Homo sapiens
  REGION 1..152: note = misc_feature - human IL-7
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 90 - protein, 157 aa, Homo sapiens
  REGION 1..157: note = misc_feature - human IL-18
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 91 - protein, 138 aa, Homo sapiens
  REGION 1..138: note = misc_feature - human IL-21
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 92 - protein, 198 aa, synthetic construct
  REGION 1..198: note = Synthetic: anti-hu-CLDN VH-CH version 1
QVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQT

SEQ ID NO: 93 - protein, 220 aa, synthetic construct
  REGION 1..220: note = Synthetic: anti-hu-CLDN VL-CL version 1
DIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 219 aa, synthetic construct
  REGION 1..219: note = Synthetic: anti-hu-CLDN VH-CH version 2QV
QLQQPGAELVRPGASVKLSCKASGYTFTSYWINWVKQRPGQGLEWIGNIYPSDSYTNYNQKFKDKATLTVDKSSSTAYMQLSSPTSEDSAVYYCARVYYGNSFAYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 95 - protein, 240 aa, synthetic construct
  REGION 1..240: note = Synthetic: anti-hu-CLDN VL-CL version 2
METDTLLLWVLLLWVPGSTGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYFYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 197 aa, Homo sapiens
  REGION 1..197: note = misc_feature - human IL-12P35 subunit mutein 1 (no signal peptide)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTAIKLCILLHAFRIRAVTIDRVMSYLNAS

SEQ ID NO: 97 - protein, 197 aa, Homo sapiens
  REGION 1..197: note = misc_feature - human IL-12P35 subunit mutein 2 (no signal peptide)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 98 - protein, 197 aa, Homo sapiens
  REGION 1..197: note = misc_feature - human IL-12P35 subunit mutein 3 (no signal peptide)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 99 - protein, 197 aa, Homo sapiens
  REGION 1..197: note = misc_feature - human IL-12P35 subunit mutein 4 (no signal peptide)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFHKTHIKLCILLHAFRIRAVTIDRVMSYLNAS

SEQ ID NO: 100 - protein, 30 aa, synthetic construct
  REGION 1..30: note = Synthetic: Non-cleavable linker
  REGION 1..30: note = misc_feature - "Gly Gly Ser" repeats at least one time and up to 10 times
GGSGGSGGSGGSGGSGGSGGSGGSGGSGGS

(19) United States
(12) Patent Application Publication
(10) Pub. No.: US 2023/0035308 A1
(43) Pub. Date: Feb. 2, 2023

(54) ACTIVATABLE MONOMERIC CYTOKINE-SINGLE CHAIN (SC) FC FUSION PROTEINS AND USES THEREOF

(71) Applicant: Aetio Biotherapy, Inc., Dallas, TX (US)

(72) Inventors: Yang Wang, Dallas, TX (US); Tao Yue, Coppell, TX (US)

(21) Appl. No.: 17/810,422

(22) Filed: Jul. 1, 2022

Related U.S. Application Data
(60) Provisional application No. 63/217,808, filed on Jul. 2, 2021.

(51) Int. Cl. C07K 14/54; A61P 35/00; C07K 16/28; C07K 14/715; C07K 14/56

(52) U.S. Cl. CPC C07K 14/5434; C07K 14/5443; A61P 35/00; C07K 16/2818; C07K 14/7155; C07K 14/56; C07K 2319/30; A61K 38/00

(57) ABSTRACT

The present invention includes compositions and methods for making and using the next generation of activatable pro-cytokine drugs by using a single-chain fragment crystallizable (Fc) region. This single-chain Fc fusion protein is engineered according to the formulas: P1-L-Fc-L-P2-L-Fc, or P1-L-Fc-L-P2-L-Fc-L-P3 in a linear sequence from amino- to carboxy-terminus, wherein protein (P) is the biologically active moiety, e.g., an antibody, or an antigen-binding fragment, or a cytokine receptor, or a cytokine, or a fusion protein consisting of above-mentioned components; L is optional and a protease cleavable, or non-cleavable linker (up to 50-mers amino acids) that is processed by enzymes enriched in tumor tissues, wherein P1, P2, and P3 are different biologically active moieties, but at least one of them is a cytokine that forms an intramolecular heterodimer. Processing the cleavable linker by tumor-specific enzymes serves as a switch to activate the cytokine in the tumor microenvironment.

### CROSS-REFERENCE TO RELATED APPLICATIONS

This application claims priority to U.S. Provisional Application Ser. No. 63/217,808, filed Jul. 2, 2021, the entire contents of which are incorporated herein by reference.

### TECHNICAL FIELD OF THE INVENTION

The present invention relates in general to the field of fusion proteins, and more particularly, to activatable monomeric cytokine-single chain (SC) Fc fusion proteins and uses thereof.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Jun. 27, 2022, is named AEBI1004.xml and is 163,866 bytes in size.

### BACKGROUND OF THE INVENTION

Without limiting the scope of the invention, its background is described in connection with activatable cytokines.

U.S. Pat. No. 10,881,741, issued to Alvarez, et al., and entitled, “Single chain Fc fusion proteins” is said to teach novel, single-chain Fc (scFc) fusion proteins having improved properties. The invention is said to include single chain fusions of soluble proteins fused to the Fc region of an immunoglobulin via a novel linker comprising a constant region of an immunoglobulin light chain linked to a CH1 constant region of an immunoglobulin heavy chain.

U.S. Pat. No. 9,856,468, issued to Salas, et al., is entitled, “Processable single chain molecules and polypeptides made using same”, and is said to teach nucleic acid molecules the encode polypeptides comprising a single chain Fc region and the polypeptides they encode. The Fc moieties of these constructs are linked by a cleavable scFc linker that is adjacent to at least one enzymatic cleavage site, e.g., an intracellular processing site. The resulting processed molecules comprise two polypeptide chains and substantially lack the extraneous amino acid sequence found in single-chain Fc linker molecule.

However, despite these advances low toxicity of novel cytokine fusion proteins and constructs designs are needed.

### SUMMARY OF THE INVENTION

The present invention addressed for the first time a number of problems with existing technologies. As described/summarized herein, the present invention includes, for example, one of more of the following features: (1) protease cleavable linker and blocking unit(s) are included to achieve limited activity of the cytokines in normal tissue and be able to be cleaved off by tumor-associated proteases so that the cytokine is released and be reactivated only at a tumor sit, (2) in addition to wild-type cytokines, cytokine muteins with reduced activities could be used in fusion protein constructs to achieve lower toxicity in circulation and or to block corresponding receptor(s)-mediated signaling activities in the treatment of autoimmune diseases, and/or (3) targeting element(s) could be included in constructs on targeting either tumor site or effective cells for further reduced toxicity or enhanced efficacy. Those multi-binding prodrugs in a single-molecule form could greatly simplify the complicated processes of production and purification.

As embodied and broadly described herein, an aspect of the present disclosure relates to an activatable monomeric cytokine-scFc fusion protein comprising: in a linear sequence from amino to carboxy terminus: a P1-L-Fc-L-P2-L-Fc fusion protein; or a P1-L-Fc-L-P2-L-Fc-L-P3 fusion protein; wherein P1, P2, and P3 are each a biologically active moiety, or portion thereof; wherein L is a cleavable or non-cleavable linker that is cleaved by enzymes enriched in tumor tissues and L is also optional (some fusion proteins with L, others may not with L); and wherein Fc is an antibody Fc fragment. In one aspect, the biologically active moiety is an antibody, or an antigen-binding fragment, a cytokine receptor, a cytokine, or combinations thereof. In another aspect, the cleavable or non-cleavable linker is up to 50 amino acids in length. In another aspect, the cleavable linker is cleaved by enzymes enriched in tumor tissues in vivo. In another aspect, P1, P2, and P3 are different biologically active moieties, but at least one of them is a cytokine. In another aspect, at least one of the P1, P2, P3, Fc, and L are human sequences. In another aspect, at least one of P1, P2, or P3, is a tumor-targeting antibody or an antigen-binding fragment, a cytokine receptor subunits or subunits that increase or reduce a cytokine activity of the fusion protein before cleavage of the linker, or a cytokine subunit that forms an intramolecular heterodimer. In another aspect, cleavage of the cleavable linker by tumor-specific enzymes activates the activable fusion protein in a tumor microenvironment. In another aspect, the antibody or antigen-binding fragment at least one of: specifically targets the fusion protein to tumor tissues, or activates residing effector cells for cancer treatment. In another aspect, the Fc fragments form a dimeric Fc region by inter-moiety disulfide bonds between cysteines in a hinge region that hold the dimeric Fc region is a functional unit to block the activity of any one of P1, P2, or P3. In another aspect, the activable fusion protein further comprises one or more protein domains at an amino-, a carboxy-, or both the amino- and carboxy-terminus of the fusion protein, wherein the one or more protein domains is an antibody, binding fragments thereof, Fc region, cytokine or receptor. In another aspect, the fusion protein comprises one or more split domains of an antibody, a cytokine, or a receptor linked with cleavable or non-cleavable linkers. In another aspect, the Fc is a wild-type or a mutated Fc. In another aspect, the cleavable linker is cleaved by a protease selected from matrix metallopeptidase (MMP)-1, MMP2, MMP3, MMP7, MMP9, MMP 10, MMP 11, MMP12, MMP13, MMP14, MMP15, MMP16, MMP17, MMP19, MMP20, MMP21, uPA, FAPa, or Cathepsin B. In another aspect, the cleavable linker is cleaved by a caspase selected from Caspase 1, Caspase 2, Caspase 3, Caspase 4, Caspase 5, Caspase 6, Caspase 7, Caspase 8, Caspase 9, Caspase 10, Caspase 11 and Caspase 12. In another aspect, the cleavable linker is cleaved by matrix metallopeptidase 14. In another aspect, the P1, P2, or P3 is a cytokine or a mutein that comprises one or more mutations with increased or reduced binding affinity to its cognate receptor. In another aspect, the biologically active moiety comprises a tumor-targeting antibody binding portion, wherein an antigen is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein. In another aspect, the e biologically active moiety comprises an antibody binding portion specific to a T-cell marker selected from CTLA-4, PD-1, Lag3, S15, B7H3, B7H4, TCR-alpha, TCR-beta, or TIM-3. In another aspect, the biologically active moiety comprises a target-binding portion specific to a T-cell activator selected from CD3, 41BB, or OX40. In another aspect, the biologically active moiety comprises an antibody binding portion specific to an antigen-presenting cell marker selected from PD-L1, CD40, CD24, B7H3, TGF-beta receptor, TNFR family members 1 to 20, CD80, CD86, FLT3, CD11c, CD8-alpha, 5B6 (CLEC9A), CD1c, CD11b, CD13, CD33, HLA-DR, CD141, CD1a, CD32, CD45, CD80, CD86, CD207, CD2, CD7, CD45RA, CD68, CD123, CD303, or CD304. In another aspect, the at least one of the biologically active moieties comprises: IL-1, IL-2, IL-4, IL-5, IL-6, IL-7, IL-8, IL-10, IL-11, IL-12, IL-13, IL-15, IL-18, IL-21, interferons α, β, or γ, colony-stimulating factors (CSFs), granulocyte-macrophage CSF, tumor necrosis factor alpha, or tumor necrosis factor beta. In another aspect, a cancer is selected from the group consisting of: acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. In another aspect, a wild-type or mutant cytokine is used in the activable fusion protein. In another aspect, the activable fusion protein has at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

As embodied and broadly described herein, an aspect of the present disclosure relates to a cancer or an autoimmune disease patient providing a subject in need thereof with an activatable monomeric cytokine-scFc fusion protein comprising: providing the cancer or autoimmune disease patient with an effective amount of an activatable monomeric cytokine-scFc fusion protein comprising a linear sequence from amino to carboxy terminus: a P1-L Fc-L-2P-L-Fc fusion protein; or a P1-L-Fc-L-P2-L-Fc-L-P3 fusion protein; wherein P1, P2, and P3 are each a biologically active moiety, or portion thereof, wherein L is a cleavable or non-cleavable linker that is cleaved by enzymes enriched in tumor tissues; and wherein Fc is an antibody Fc fragment. In one aspect, the biologically active moiety is an antibody, or an antigen-binding fragment, or a cytokine receptor, a cytokine, or combinations thereof. In another aspect, the cleavable or non-cleavable linker is up to 50 amino acids in length. In another aspect, the cleavable linker is cleaved by enzymes enriched in tumor tissues in vivo. In another aspect, P1, P2, and P3 are different biologically active moieties, but at least one of them is a cytokine. In another aspect, at least one of the P1, P2, P3, Fc, and L are human sequences. In another aspect, at least one of P1, P2, or P3, is a tumor-targeting antibody or an antigen-binding fragment, a cytokine receptor subunits or subunits that increase or reduce a cytokine activity of the fusion protein before cleavage of the linker, or a cytokine subunit that forms an intramolecular heterodimer. In another aspect, the cleavage of the cleavable linker by tumor-specific enzymes activates the activable fusion protein in a tumor microenvironment. In another aspect, the antibody or antigen-binding fragment at least one of it facilitates the fusion protein specifically targeting on tumor tissues, or activates residing effector cells for cancer treatment. In another aspect, the Fc fragments form a dimeric Fc region by inter-moiety disulfide bonds between cysteines in a hinge region that hold the dimeric Fc region is a functional unit to block the activity of any one of P1, P2, or P3. In another aspect, the method further comprises adding one or more protein domains at an amino-, a carboxy-, or both the amino- and carboxy-terminus of the fusion protein, wherein the one or more protein domains is an antibody, binding fragments thereof, Fc region, cytokine or receptor. In another aspect, the fusion protein comprises one or more split domains of an antibody, a cytokine, or a receptor linked with cleavable or non-cleavable linkers. In another aspect, the Fc is a wild-type or a mutated Fc. In another aspect, the cleavable linker is cleaved by a protease selected from matrix metallopeptidase-1 (MMP1), MMP2, MMP3, MMP7, MMP9, MMP 10, MMP 11, MMP 12, MMP 13, MMP 14, MMP 15, MMP16, MMP17, MMP19, MMP20, MMP21, uPA, FAPa, or Cathepsin B. In another aspect, the cleavable linker is cleaved by a caspase selected from Caspase 1, Caspase 2, Caspase 3, Caspase 4, Caspase 5, Caspase 6, Caspase 7, Caspase 8, Caspase 9, Caspase 10, Caspase 11 and Caspase 12. In another aspect, the cleavable linker is cleaved by matrix metallopeptidase 14. In another aspect, the P1, P2, or P3 is a cytokine that comprises one or more mutations to reduce binding affinity to its cognate receptor. In another aspect, the biologically active moiety comprises a tumor-targeting antibody binding portion, wherein an antigen is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein. In another aspect, the biologically active moiety comprises an antibody binding portion specific to a T-cell marker selected from CTLA-4, PD-1, Lag3, S15, B7H3, B7H4, TCR-alpha, TCR-beta, or TIM-3. In another aspect, the biologically active moiety comprises a target-binding portion specific to a T-cell activator selected from CD3, 41BB, or OX40. In another aspect, the biologically active moiety comprises an antibody binding portion specific to an antigen-presenting cell marker selected from PD-L1, CD40, CD24, B7H3, TGF-beta receptor, TNFR family members 1 to 20, CD80, CD86, FLT3, CD11c, CD8-alpha, 5B6 (CLEC9A), CD1c, CD11 b, CD13, CD33, HLA-DR, CD141, CD1a, CD32, CD45, CD80, CD86, CD207, CD2, CD7, CD45RA, CD68, CD123, CD303, or CD304. In another aspect, at least one of the biologically active moiety comprises: IL-1, IL-2, IL-4, IL-5, IL-6, IL-7, IL-8, IL-10, IL-11, IL-12, IL-13, IL-15, IL-18, IL-21, interferons α, β, or γ, colony-stimulating factors (CSFs), granulocyte-macrophage CSF, tumor necrosis factor alpha, or tumor necrosis factor beta. In another aspect, the cancer is selected from the group consisting of: acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. In another aspect, the autoimmune disease is a human or animal autoimmune disease selected from autoimmune diseases or disorders include, but are not limited to, inflammatory responses such as inflammatory skin diseases including psoriasis and dermatitis (e.g., atopic dermatitis); responses associated with inflammatory bowel disease (such as Crohn's disease and ulcerative colitis); dermatitis; allergic conditions such as eczema and asthma; rheumatoid arthritis; systemic lupus erythematosus (SLE) (including but not limited to lupus nephritis, cutaneous lupus); diabetes mellitus (e.g., type 1 diabetes mellitus or insulin-dependent diabetes mellitus); multiple sclerosis and juvenile-onset diabetes. In another aspect, a wild-type or mutant cytokine is used in the activable fusion protein. In another aspect, the activable fusion protein has at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

As embodied and broadly described herein, an aspect of the present disclosure relates to an activatable monomeric cytokine-scFc fusion protein comprising: in a linear sequence from amino to carboxy terminus: a P1-L-Fc-L-2P-L-Fc fusion protein; or a P1-L-Fc-L-P2-L-Fc-L-P3 fusion protein; wherein P1, P2, and P3 are each a biologically active moiety, wherein L is a cleavable or non-cleavable linker that is cleaved by enzymes enriched in tumor tissues; and wherein Fc is an antibody Fc fragment. In one aspect, the nucleic acid encodes an activable fusion protein with at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

As embodied and broadly described herein, an aspect of the present disclosure relates to a vector that expresses a nucleic acid that encodes an activable monomeric cytokine-scFc fusion protein comprising: in a linear sequence from amino to carboxy terminus: a P1-L-Fc-L-2P-L-Fc fusion protein; or a P1-L-Fc-L-P2-L-Fc-L-P3 fusion protein; wherein P1, P2, and P3 are each a biologically active moiety, wherein L is a cleavable or non-cleavable linker that is cleaved by enzymes enriched in tumor tissues; and wherein Fc is an antibody Fc fragment. In another aspect, the activable fusion protein has at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

As embodied and broadly described herein, an aspect of the present disclosure relates to a host cell that comprises a vector that expresses a nucleic acid that encodes an activatable monomeric cytokine-scFc fusion protein comprising: in a linear sequence from amino to carboxy terminus: a P1-L-Fc-L-2P-L-Fc fusion protein; or a P1-L-Fc-L-P2-L-Fc-L-P3 fusion protein; wherein P1, P2, and P3 are each a biologically active moiety, wherein L is a cleavable or non-cleavable linker that is cleaved by enzymes enriched in tumor tissues; and wherein Fc is an antibody Fc fragment. In another aspect, the activable fusion protein has at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

As embodied and broadly described herein, an aspect of the present disclosure relates to a method of making an activatable monomeric cytokine-scFc fusion protein comprising: expressing a nucleic acid that encodes an activatable monomeric cytokine-scFc fusion protein comprising: in a linear sequence from amino to carboxy terminus: a P1-L-Fc-L-2P-L-Fc fusion protein; or a P1-L-Fc-L-P2-L-Fc-L-P3 fusion protein; wherein P1, P2, and P3 are each a biologically active moiety, wherein L is a cleavable or non-cleavable linker that is cleaved by enzymes enriched in tumor tissues; and wherein Fc is an antibody Fc fragment. In another aspect, the activable fusion protein has at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

As embodied and broadly described herein, an aspect of the present disclosure relates to an engineered human IL-12P35 subunit mutein that does not include a signal peptide, wherein the residues that substitute residues 158 K, 162 E, 163 E, 165 D, 167 Y, 168 K, 170 K, and 172 K, are selected from A, H, D, E, R, and K, as applicable. For example, 158 K is substituted with A, H, D, E, or R; 162 E is substituted with A, H, D, R, or K;163 E is substituted with A, H, D, R, or K; 165 D is substituted with A, H, E, R, or K; 167 Y is substituted with A, H, D, E, R, or K; 168 K is substituted with A, H, D, E, or R; 170 K is substituted with A, H, D, E, or R; and 172 K is substituted with A, H, D, E, or R.

### BRIEF DESCRIPTION OF THE DRAWINGS

For a more complete understanding of the features and advantages of the present invention, reference is now made to the detailed description of the invention along with the accompanying figures and in which:

For a more complete understanding of the features and advantages of the present invention, reference is now made to the detailed description of the invention along with the accompanying figures and in which:

FIGS. 1A and 1B are illustrations of exemplary monomeric cytokine-scFc fusion proteins of the invention with cleavable or non-cleavable linkers to connect the biologically active moiety and Fc region. Linkers are optional in all fusion proteins in the present invention. P1, P2 and P3 is the biologically active moiety, which comprises an antibody, or an antigen-binding fragment, or a cytokine receptor, or a cytokine, or a fusion protein from above-mentioned components. Fc is fragment crystallizable region

FIGS. 2A to 2C illustrate IL-15-scFc fusion proteins and their cleavage. IL-15Ra-Linker(L)-Fc-L-IL-15-L-Fc (1156) is indicated in FIG. 2A, IL-15Rb-L-IL-15Ra-L-Fc-L-IL-15-L-Fc (1198B) is indicated in FIG. 2B, and IL-15Ra-L-Fc-L-IL-15-L-Fc-L-Rb (1199C) is indicated in FIG. 2C. Cleavage of IL-15-scFc fusion protein with mmp14 enzyme release IL15 from the linkage of one side of Fc and removes inhibitory IL-15 receptor β, which allows most activated complex formation of IL-15 receptor α with IL-15.

FIG. 3A illustrates pro-drug activation cleavage-mediated by MMP14- digestion of IL-15-scFc fusion proteins in vitro. SDS gel analysis of 1156+/− mmp14 & 1198B+/− mmp14 at reducing (R) and non-reducing (NR) condition. FIG. 3B illustrates IL-15 activity in 1198B increased after cleavage with mmp14, and the activity was measured via HEKblue-IL15 assay. FIGS. 3C and 3D illustrate that monomeric IL-15-scFc fusion protein exhibited even higher activity than homo-dimeric IL-15Ra-IL-15-Fc fusion protein (1100) in vitro and in vivo, and the activities were measured by HEKblue-IL15 assay and humanized mouse tumor growth model.

FIGS. 4A, 4B, and 4C illustrate three proteins derived from IL-15-scFc fusion protein (1199C). Their P1 moieties are comprising anti-human PD1 antibody scFv fused to IL-15Rα (1225), IFNα2m- fused to IL-15Rα (1226), and anti-human CTLA4 antibody scFv fused to IL-15Rα (1232), respectively.

FIG. 5A illustrates the expression and cleavage of different IL-15-scFc fusion proteins, including 1156, 1198, 1199, 1225, 1226, and 1232, analyzed by SDS-PAGE at reducing (R) and non-reducing (NR) condition. FIG. 5B illustrates IL-15 activities from different IL-15-scFc fusion proteins, increased after cleavage with mmp14, and the activities were measured via HEKblue-IL15 assay. FIG. 5C, illustrates IL-15 activities from different IL-15-scFc fusion proteins increased after cleavage with mmp14, and the activities were revealed by IFN gamma production from hu PBMC after stimulated with IL-15-scFc fusion proteins.

FIGS. 6A to 6F illustrate different monomeric IL-12-scFc fusion proteins fused to antibodies, and/or IL-12 receptor subunits of IL12Rβ1 and IL12Rβ2. IL-12 is a heterodimeric cytokine that includes two subunits: P35 and P40. Interleukin 12 receptor is a type 1 cytokine receptor, consisting of beta 1 and beta 2 subunits that bind to interleukin 12. The positions of the biologically active moieties are interchangeable. FIG. 6A shows IL-12p40-L-Fc-L-IL-12p35-L-Fc (IT25). FIG. 6B shows IL12β1-IL-12p40-L-Fc-L-IL-12p35-L-Fc (IT30). FIG. 6C shows IL-12p40-L-Fc-L-IL-12p35-L-Fc-L-IL12R132 (IT32). FIG. 6D shows IL12Rβ1-IL-12p40-L-Fc-L-IL-12p35-L-Fc-L-IL12Rβ2 (IT33). FIG. 6E shows anti-hPD 1(V_{L}-VH)-L-IL-12p40-L-Fc-L-IL-12p35-L-Fc-L-IL12Rβ2. FIG. 6F shows anti-hPD 1 (V_{L}-VH)-L-IL-12p40-L-Fc-L-IL-12p35-L-Fc.

FIG. 7A illustrates pro-drug activation cleavage-mediated by MMP14 digestion of monomeric IL-12-scFc fusion proteins of IT30,32,33. SDS gel analysis of non-cleaved or cleaved proteins at reducing (R) and non-reducing (NR) conditions. Rb1 is cut into two pieces marked as Rβ 1.1 and Rβ 1.2 from IT30 & 33; Rβ2 is cut out as one fragment from IT32. FIG. 7B illustrates IL-12 activity increased after cleavage with mmp14 measured via HEKblue-IL12 assay.

FIGS. 8A, 8B and 8C illustrate other three exemplary monomeric cytokine scFc fusion proteins. FIG. 8A is an Anti-huPDL1(Fab)-L-Fc-IFNα2m-L-Fc-L-anti-huCTLA4 (scfv) fusion protein. FIG. 8B shows an Anti-huPD1(scFv)-L-Fc-L-IL-2-L-Fc-L-Ra fusion protein. FIG. 8C shows a Rα-L-Fc-L-IL-18-L-Fc-L-Rβ fusion protein.

FIGS. 9A and 9B are graphs that illustrate the effect of different constructs. FIG. 9A illustrates that monomeric IL-12p40-Fc-L-IL-12p35-mutein1-Fc fusion protein exhibited lower activities than monomeric IL-12p40-Fc-L-IL-12p35-wild type-Fc (IT25) fusion protein in vitro, and the activities were measured by HEKblue-IL12 assay. 9B illustrates that monomeric IL-12p40-Fc-L-IL-12p35-mutein1/2/3-Fc fusion proteins exhibited comparable binding affinities compared with monomeric IL-12p40-Fc-L-IL-12p35-wild type-Fc (IT25) fusion protein in vitro, and the binding affinities were measured by flow cytometry using recombinant monomeric IL-12p40-Fc-L-IL-12p35-mutein-Fc fusion proteins at a series of concentrations stain on 293 cells expressing IL-12 receptor complex. The IL-12 mutein protein sequences are indicated in SEQ ID NOS: 96-99.

FIGS. 10A and 10B show the results from using different constructs shown in FIG. 10B. FIG. 10A illustrates that target-binding polypeptide (anti-CLDN)-fused IL-12p40-Fc-L-IL-12p35-wild type-Fc inhibits tumor growth in the mouse tumor model. Pro-anti-CLDN-IL-12p40-Fc-L-IL-12p35-wild type-Fc drug at the low concentration significantly inhibits tumor growth in the humanized mouse tumor model, compared with IL-12p40-Fc-L-IL-12p35-wild type-Fc IL-2 treatment. NSG-SGM3 mice were inoculated with A549 expressing CLDN18.2 protein, followed by huPBMC transfer, and the treatment with indicated drugs. The IL-12 mutein protein sequences are indicated in SEQ II) NO:96-99, and the anti-human CLDN18.2 antibody sequences are indicated in SEQ II) NO:92-95. FIG. 10B illustrates illustrate IL-12p40-Fc-L-IL-12p35-wild type/mu-Fc and anti-CLDN-IL-12p40-Fc-L-IL-12p35-wild type-Fc fusion proteins.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

High-dose cytokines, used in the treatment of cancers, are often associated with significant side effects due to their toxicity in multiple organ systems, including the heart, lungs, kidneys, and central nervous system. It is critical to de-activate cytokine(s) activity as much in peripheral organs and specifically re-activate cytokine(s) mainly in the tumor microenvironment, to reduce its systemic toxicity. The inventors have designed the next generation of pro-cytokine drugs by using a single chain fragment crystallizable (Fc) region. This single-chain Fc fusion protein contains at least two Fc moieties which form a dimeric Fc region through inter-moiety disulfide bonds between cysteines in the hinge region to hold the two Fc moieties together and create a functional unit to potentially alter the activity of the fused cytokine. A fusion protein is engineered according to the formulas:

P1-L-Fc-L-P2-L-Fc, or

P 1-L-Fc-L-P2-L-Fc-L-P3

in a linear sequence from amino- to carboxy-terminus, wherein protein (P) is the biologically active moiety (or portion thereof), which can be an antibody, or an antigen-binding fragment, or a cytokine receptor, or a cytokine, or a fusion protein consisting of above-mentioned components; and L is optional, and when present is either a protease cleavable or non-cleavable linker (up to 50-mers amino acids). The protease cleavable linker is able to be processed (cut) by protease enzymes enriched in tumor tissues. Fc is the Fc region of an antibody. Wherein P1, P2, and P3 are different biologically active moieties, but at least one of them is a cytokine (or mutant cytokine), and the rest could be a tumor-targeting antibody or an antigen-binding fragment, or the corresponding receptor subunits(s) /or peptides that reduce the cytokine activity, or the other cytokine subunit to form an intramolecular heterodimer to increase cytokine activity. Processing the cleavable linker by tumor-specific enzymes serves as a switch to activate the cytokine in the tumor microenvironment. Antibody or antigen-binding fragment in cytokine-scFc fusion protein specifically targets it to tumor tissues and activates cytokine activity on residing effector cells for cancer eradication.

U.S. Pat. No. 9,856,468 B2 teaches a composition for treating or preventing a disease or disorder in a subject, where the disease or disorder is a clotting disorder, a neurological disorder, an inflammatory disorder, an autoimmune disorder, and a neoplastic disorder, preferably a disorder affecting hemostasis. The present invention overcomes critical problems with the prior art, including: (1) the cytokine-scFc fusion protein of the present invention is activable, and the activity of the cytokine is inhibited in the normal tissue due to steric hindrance by either dimerized Fc moieties, or the corresponding cytokine receptor (peptides). The unprocessed fusion protein is under de-activated status and re-activated in the tumor tissue by enzyme-medicated cleavage occurring to the linker region of the pro-drugs. (2) The two wild type or mutated Fc moieties are used in the fusion protein of the present invention to more potently activate T cells to kill tumor cells, alternatively, the two mutant Fcs that fail to bind FcR on phagocytes are used to reduce the depletion of effector cells. (3) The present invention uses novel engineered mutations to increase the binding affinity between the cytokine and its receptor to enhance the blocking efficacy of prodrug. (4) The antibody-fused cytokine of the present invention selectively targets the activity of the fusion protein to the tumor tissue. (5) While antibody and cytokine can form various combinational heterodimeric or hetero-multimeric monomers, using single chained two Fc s form disulfide bonds internally in similar to traditional homodimers instead of heterodimers (knob and hole format).

U.S. Pat. No. 10,881,741 B2 teaches fragment crystallizable fusion proteins is used for treating or preventing disease or disorder, e.g., cancer, inflammatory diseases, arthritis, osteoporosis, hepatitis, and bacterial infections. The present invention overcomes critical problems with this prior art, including: (1) The cytokine-scFc fusion protein of the present invention includes at least two Fc domains, which form a homodimeric Fc region through inter-moiety disulfide bonds between cysteines in the hinge region to hold the two Fc moieties together and create a functional unit to potentially block the activity of the fused cytokine. By contrast, U.S. Pat. No. 10,881,741 only uses one Fc moiety that will either act as a monomer or form a homodimer through intermolecular interaction. (2) The cytokine-scFc fusion protein of the present invention is activable, and the activity of the cytokine is inhibited in the normal tissue due to steric hindrance by either dimerized Fc moieties, or the corresponding cytokine receptor(s). The fusion protein is processed and re-activated in the tumor tissue by enzyme-medicated cleavage occurring to the linker region of the pro-drugs, thus reducing or eliminating off-target activation of the cytokine.

Thus, the activatable fusion proteins of the present invention have several distinct advantages. First, the cytokine-scFc fusion protein is activable, and the activity of the cytokine is blocked in the normal tissue due to steric hindrance by either dimerized Fc moieties, or the corresponding cytokine receptor. The unprocessed fusion protein is activated in the tumor tissue by enzyme-medicate cleavage occurring to the linker region of the pro-drugs. Second, the inventors engineered novel mutations to increase the binding affinity between the cytokine and its receptor to enhance the efficacy of prodrugs. Third, wild-type or mutated Fc moiety are used in the activatable fusion protein to more potently activate T cells and kill tumor cells, alternatively, the mutant Fc fails to bind FcR on phagocytes and reduces the depletion of effector cells. Fourth, the antibody-fused cytokine selectively targets the activity of the fusion protein to the tumor tissue by either recognizing tumor-specific antigens, or antigens expressing on tumor-infiltrating T cells. Finally, when compared with the conventional approach for the heterodimeric Ab-cytokine-Fc production, the monomeric cytokine-scFc homodimer fusion protein of the present invention improves manufacturability, and usually leads to increased product yield or purity and reduced cost.

As used herein, the terms “polypeptide” refers to an amino acid chain, regardless of length or post-translational modification (e.g., glycosylation or phosphorylation). Polypeptides can include full length proteins or fragments or variants thereof. A “polypeptide of interest” refers to a target sequence expressed at cell, as described herein. In some embodiments, a polypeptide of interest can be a polypeptide that is not expressed in nature in the relevant type of cell or is not expressed at the level that the polypeptide is expressed when expression is achieved by intervention of the hand of man, as described herein. In certain embodiments, a polypeptide of interest can include sequences that are not naturally found in the relevant cell but are found naturally in other cell types or organisms.

As used herein, the term “fusion protein” refers to a hybrid protein, that includes portions of two or more different polypeptides, or fragments thereof, resulting from the expression of a polynucleotide that encodes at least a portion of each of the two polypeptides. Non-limiting examples of the activatable monomeric cytokine-scFc fusion protein of the present invention include fusion proteins having the general formula: in a linear sequence from amino to carboxy terminus: a P1-L-Fc-L-P2-L-Fc fusion protein; or a P1-L-Fc-L-P2-L-Fc-L-P3 fusion protein; wherein P1, P2, and P3 are each a biologically active moiety, or portion thereof; wherein L is a cleavable or non-cleavable linker that is cleaved by enzymes enriched in tumor tissues; and wherein Fc is an antibody Fc fragment.

As used herein, the terms “nucleic acid,” “polynucleotide,” and “oligonucleotide” are used interchangeably herein to refer to a polymer of at least three nucleotides. A nucleoside comprises a nitrogenous base linked to a sugar molecule. In a polynucleotide, phosphate groups covalently link adjacent nucleosides to form a polymer. The polymer can include natural nucleosides (e.g., adenosine, thymidine, guanosine, cytidine, uridine, deoxyadenosine, deoxythymidine, deoxyguanosine, and deoxycytidine), nucleoside analogs, chemically modified bases, biologically modified bases (e.g., methylated bases), intercalated bases, and/or modified sugars (e.g., modified purines or pyrimidines). See, Romberg and Baker (1992) DNA Replication, 2nd Ed., Freeman, San Francisco, Calif.; Scheit (1980) Nucleotide Analogs, John Wiley, New York, N.Y.), and U.S. Patent Publication No. 20040092470 and references therein for further discussion of various nucleotides, nucleosides, and backbone structures that can be used in the polynucleotides described herein. A polynucleotide can have any length and sequence and can be single-stranded or double-stranded. Where this document provides a nucleic acid sequence, the complementary sequence also is provided. Further, where a sequence is provided as DNA, the corresponding RNA sequence (i.e., the sequence in which T is replaced by U) also is provided.

As used herein, the term “nucleic acid construct” refers to a nucleic acid that has been recombinantly modified or is derived from such a nucleic acid. For example, a nucleic acid construct can contain a mutation, deletion, or substitution relative to a naturally occurring nucleic acid molecule. A nucleic acid construct can comprise two or more nucleic acid segments that are derived from or originate from different sources such as different organisms (e.g., a recombinant polynucleotide). The sequence of one or more portions of a nucleic acid construct may be entirely invented by man.

As used herein, the “nucleic acid sequence” refers to the nucleic acid material itself and is not restricted to the sequence information (i.e., the succession of letters chosen among the five base letters A, G, C, T, or U) that biochemically characterizes a specific nucleic acid (e.g., DNA or RNA) molecule.

As used herein, the term “gene” has its meaning as understood in the art. In general, the term “gene” refers to a nucleic acid that includes a portion encoding a protein; the term optionally may encompass regulatory sequences such as promoters, enhancers, terminators, etc., in addition to coding sequences (open reading frames). This definition is not intended to exclude application of the term “gene” to non-protein coding expression units but rather to clarify that, in most cases, the term as used in this document refers to a protein-encoding nucleic acid. It will be appreciated that the definition of gene can include nucleic acids that do not encode proteins, but rather provide templates for transcription of functional RNA molecules such as tRNAs or rRNAs, for example.

As used herein, the terms “gene product” or “expression product” refer to, in general, an RNA transcribed from the gene, or a polypeptide encoded by an RNA transcribed from the gene. Expression of a gene or a polynucleotide refers to (1) transcription of RNA from the gene or polynucleotide; (2) translation of RNA transcribed from the gene or polynucleotide, or both (1) and (2).

As used herein, the term “vector” refers to a nucleic acid or a virus, viral genome, plasmid, or portion thereof that is a nucleic acid molecule that can replication and/or express a nucleic acid molecule in cell. Where the vector is a nucleic acid, the nucleic acid molecule to be transferred is generally linked to, e.g., inserted into, the vector nucleic acid molecule. A nucleic acid vector may include sequences that direct autonomous replication within suitable host cells (e.g., an origin of replication), or may include sequences sufficient to allow integration of part of all of the nucleic acid into host cell DNA. Useful nucleic acid vectors include, for example, DNA or RNA plasmids, cosmids, and naturally occurring or modified viral genomes or portions thereof, or nucleic acids (DNA or RNA) that can be packaged into viral capsids. Plasmid vectors typically include an origin of replication and one or more selectable markers. Plasmids may include part or all of a viral genome (e.g., a viral promoter, enhancer, processing or packaging signals, etc.). Viruses or portions thereof (e.g., viral capsids) that can be used to introduce nucleic acid molecules into cells are referred to as viral vectors. Useful animal viral vectors include adenoviruses, retroviruses, lentiviruses, vaccinia virus and other poxviruses, herpes simplex virus, and others. Useful plant viral vectors include those based on to bamoviruses, ilarviruses, etc. Viral vectors may or may not contain sufficient viral genetic information for production of infectious virus when introduced into host cells, i.e., viral vectors may be replication-defective, and such replication-defective viral vectors may be preferable for certain embodiments of the invention. Where sufficient information is lacking it may, but need not be, supplied by a host cell or by another vector introduced into the cell.

As used herein, the term “expression vector” is a vector that includes one or more expression control sequences, and an “expression control sequence” is a DNA sequence that controls and regulates the transcription and/or translation of another DNA sequence.

As used herein, the term “operably linked” refers to a relationship between two nucleic acid sequences wherein the expression of one of the nucleic acid sequences is, e.g., controlled by, regulated by, or modulated by the other nucleic acid sequence. For example, transcription of a nucleic acid sequence is directed by an operably linked promoter sequence; post-transcriptional processing of a nucleic acid is directed by an operably linked processing sequence; translation of a nucleic acid sequence is directed by an operably linked translational regulatory sequence; transport or localization of a nucleic acid or polypeptide is directed by an operably linked transport or localization sequence; and post-translational processing of a polypeptide is directed by an operably linked processing sequence. A nucleic acid sequence that is operably linked to a second nucleic acid sequence typically is covalently linked, either directly or indirectly, to such a sequence, although any effective three-dimensional association is acceptable. It is noted that a single nucleic acid sequence can be operably linked to a plurality of other sequences. For example, a single promoter can direct transcription of multiple RNA species. A coding sequence is “operably linked” and “under the control” of an expression control sequence in a cell when RNA polymerase is able to transcribe the coding sequence into mRNA, which then can be translated into the protein encoded by the coding sequence.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein. Many of the procedures useful for practicing the present invention, whether or not described herein in detail, are well known to those skilled in the arts of molecular biology, biochemistry, immunology, and medicine.

As used herein, the term “host cell” refers to cells into which a recombinant expression vector can be introduced. A host cell for use with the disclosed expression systems and methods typically is a eukaryotic cell, such as a mammalian, insect, or plant cell. As used herein, “transformed” and “transfected” encompass the introduction of a nucleic acid molecule (e.g., a vector) into a cell by one of a number of techniques.

As used herein, the term “identity” refers to the extent to which two or more nucleic acid sequences or two or more amino acid sequences are the same. The percent identity between two sequences over a window of evaluation is computed by aligning the sequences, determining the number of nucleotides or amino acids within the window of evaluation that are opposite an identical nucleotide or amino acid, allowing the introduction of gaps to maximize identity, dividing by the total number of nucleotides or amino acids in the window, and multiplying by 100.

Percent identity for any nucleic acid or amino acid sequence is determined as follows. First, a nucleic acid or amino acid sequence is compared to the identified nucleic acid or amino acid sequence using the BLAST 2 Sequences (B12seq) program from the stand-alone version of BLASTZ containing BLASTN version 2.0.14 and BLASTP version 2.0.14, or equivalents. This stand-alone version of BLASTZ can be accessed at the U.S. government's National Center for Biotechnology Information website (World Wide Web at ncbi.nlm.nih.gov/blast/executables). Instructions explaining how to use the B12seq program can be found in the readme file accompanying BLASTZ.

In some cases, the nucleic acid or polypeptide sequence can include a sequence with at least 90 percent sequence identity (e.g., at least 90, 91, 92, 93, 94, 95, 96, 97, 98, or 99 percent sequence identity, or 100 percent sequence identity) to one or more sequences as set forth in SEQ ID NOS: 1, 4, 5, 6, 7, 8, 9, 10, 11, 12, 69, 70, 71, 72, 73, 74, 75, 76, 77, 78, or 79. In some embodiments, the fusion protein can have an amino acid sequence with at least 90 percent sequence identity (e.g., at least 90, 91, 92, 93, 94, 95, 96, 97, 98, or 99 percent sequence identity, or 100 percent sequence identity) to one or more sequences disclosed herein, however, certain specific sequences are those set forth in SEQ ID NOS: 88, 89, or 90.

Each of the activatable monomeric cytokine-scFc fusion proteins of the present invention are activated by a protease that is upregulated or secreted during a viral infection. Examples of protease include membrane anchored MMPs MMP14 (MT1-MMP), MMP15 (MT2-MMP), MMP16 (MT3-MMP), MMP17 (MT4-MMP), MMP24 (MT5-MMP), MMP25 (MT5-MMP or leukolysin), or matrilysins MMP-7 and MMP-26, or stromelysins MMP3, MMP10, MMP11, MMP19, or gelatinases MMP2, MMP9, or collagenases MMP1, MMP8, MMP13, MMP18, or any one of Caspase 1, Caspase 2, Caspase 3, Caspase 4, Caspase 5, Caspase 6, Caspase 7, Caspase 8, Caspase 9, Caspase 10, Caspase 11 and Caspase 12.
As used herein, the term “biologically active moiety” or “biologically active moieties” refer to a polypeptide, portion, fragment, or component of a polypeptide capable of performing a function, an action or a reaction in a biological context. A biologically active moiety may comprise a complete protein or biologically active portion(s) thereof. For example, the term “biologically active moiety” includes active and functional molecules, binding domains of molecules which bind to components of a biological system, such as portions of a cytokine and/or cytokine receptor. Non-limiting examples of cytokines and/or their receptors that can be used with the present invention include, e.g., at least one of the biologically active moieties comprises: IL-1, IL-2, IL-4, IL-5, IL-6, IL-7, IL-8, IL-10, IL-11, IL-12, IL-13, IL-15, IL-18, IL-21, interferons α, β, or γ, colony-stimulating factors (CSFs), granulocyte-macrophage CSF, tumor necrosis factor alpha, or tumor necrosis factor beta.

As used herein, the term “cognate receptor” refers to the receptor that is known to bind to a cytokine. For example, the cognate receptors of the IL-1 family of cytokine include: IL-1α and IL-β, IL-33, and IL-36 subfamilies share IL-1RAcP as their secondary receptor, while IL-18 subfamily utilizes a distinct secondary receptor. The cognate receptor for IL-2 is the interleukin-2 receptor (IL-2R), which is a heterotrimeric protein that includes the IL-2R alpha, beta and gamma chains. Likewise, the other cytokines (IL-1, IL-2, IL-4, IL-5, IL-6, IL-7, IL-8, IL-10, IL-11, IL-12, IL-13, IL-15, IL-18, IL-21, interferons α, β, or γ, colony-stimulating factors (CSFs), granulocyte-macrophage CSF, tumor necrosis factor alpha, or tumor necrosis factor beta) can include their cognate receptor.

As used herein, the term “scFc polypeptide” refers to a polypeptide comprising a single-chain Fc (scFc) region.

The specificity of the activatable monomeric cytokine-scFc fusion proteins of the present invention can be increased by selecting activating peptides that are susceptible to matrix metalloproteinases (MMPs) in tissues open for viral invasion. Inflamed or infected cells express higher levels of MMP. Thus, the present invention includes pro-formulations of MMP-sensitive activatable monomeric cytokine-scFc fusion proteins to target MMP enriched tissues to help reduce side-effects and off-target toxicities. The activatable monomeric cytokine-scFc fusion proteins can also compete with viruses for MMP action, which contributes to tissue damage during viral infections such as influenza. A low dose of a recombinant cytokine may not be effective (due to toxicity and short half-life), while the activatable monomeric cytokine-scFc fusion proteins of the present invention allows for a greater therapeutic index and use of higher doses in therapy.

Materials and Methods. Cell lines and reagents. The 293F cells (ThemoFisher #11625019) were grown in CD OptiCHO serum free medium (Gibco #12681-011). HEKblue -IL2 cell line was purchase from InvivoGen (#hkb-il2) and used for detecting IL15 activity according to manufacturer's protocol. HEKblue-IL12 cell line was purchase from InvivoGen (#hkb-il12) and used for detecting IL12 activity according to manufacturer's protocol. hPBMC cells were purchased from Stemcell Technologies (#700025) and cultured in assay medium [10% heated FBS (56° C.×20 min)+10 mM HEPES+100 μg/ml P/S+50 uM 2-Mercaptoethanol in DMEM]. The MDA-MB-231 breast cancer cell line and HCT116 cGAS human colon carcinoma cell line (with cGAS mutation) was maintained in DMEM supplemented with 10% FBS and 1% penicillin-streptomycin (100 units/mL-100 μg/mL). Adherent cell monolayers were cultured in tissue culture dishes for passage and experiments. Both cell lines were maintained at 37° C. in a humidified 5% carbon dioxide (CO₂) atmosphere.

SDS SurePAGE, Bis-Tris, 4-20% (GenScript #M00657), or 4-12% (GenScript #M00653).

BD Cytometric Bead Array (CBA) Human Th1/Th2 Cytokine kit II was purchased from BD Biosciences (BD #551809) and used for detecting cytokines (IL2, IL4, IL6, IL10, TNF, IFN-g) from activated human PBMC by purified fusion proteins listed in the patent.

NEBuilder® HiFi DNA Assembly Master Mix was purchased from NEB Bio (#E2621X) and used for cloning. PEI MAX®-Transfection Grade Linear Polyethylenimine Hydrochloride (MW 40,000) was purchased from PolySciences #24765-1. CaptivA rProtein A affinity Resin (Repligen #CA-PRI-0100) and used for transient transfection on protein production in 293F cells.

Molecular cloning. The DNA fragments of specific inserts were generated via PCR, or gene synthesis (Genewiz) were assembled and cloned into mammalian expression vector pEE6.4 (LifeScienceMarket #PVT1802), down-streamed to mouse Igk leader sequence, using NEBuilder® HiFi DNA Assembly Master Mix and then transformed to Top10 competent cells.

Production of fusion proteins and purification. The recombinant plasmid was transiently transfected into 293F cells via PEI. Supernatants were collected on day 4˜5 after transfection. The fusion protein was purified using a Protein A-Sepharose resin column according to the manual (Repligen Corporation).

In vitro digestion of cleavable fusion proteins. rhMMP-14 (R&D Systems #918-MPN-010) at 100 μg/mL was activated with 0.1 μg/mL of rhTrypsin in Activation Buffer (50 mM Tris, 1 mM CaCl2, 0.5% (w/v) Brij-35, pH 7.5) at 37° C. for 1 hours, then 1 mM AEBSF (1ul of 100 mM stock) was added and incubate for 15 min at room temperature (RT) to stop the reaction.

Cleavable fusion proteins was co-cultured with activated MMP14 in Assay Buffer (50 mM Tris, 3 mM CaCl2, 1 μM ZnC12, pH 8.5) at RT overnight or 37° C. for 4˜18 hours. The proteins cleavage was identified by reduced or non-reduced SDS-PAGE.

Functional IL-15 activity of fusion proteins was measured using HEKblue-IL2 cells (InvivoGen #hkb-il2), and functional IL-12 activity of fusion proteins was measured using HEKblue-IL12 cells (InvivoGen #hkb-il12). The reporter cell lines were seeded at 1×10⁵ (in 60ul assay medium) per well in 96 well plate one day before assay occurs, and cultured overnight in 37oC 5% CO2 incubator. Next day, 60 μL of serially diluted (at 1:3 or 1:4) purified proteins or MMP cleaved products were added into wells containing seeded cells in 96 well plate, continued to be cultured for 24 hours in 37° C. 5% CO2 incubator. Next day, take 20˜25ul culture supernatants from each well into a new 96-well plate, add 80ul of Quanti-blue (rep-qbs3) to each well, incubate at 37° C. from 15 min˜60 min. Absorbance was read at 650 nm.

Humanized mice and tumor models: Approximately 1˜1.5×10⁶ HCT116 cGAS or MDA-MB-231 were suspended in 100 Al of PBS and injected subcutaneously into right flank of NSG-SGM3 mice. On day 8, 7×10⁶ human CBMC cells were injected (I.V) to tumor bearing mice. Tumor volumes were measured via caliper twice a week and calculated according to the formula (length ×width ×height/2). After the tumor was established on day 16, mice were treated with various fusion proteins at equal molar amount in every 3-4 days.

Statistical analysis. Analyses were performed using GraphPad Prism version 6.0 (GraphPad Software). Data are shown as the mean±SEM. Statistical analyses were compared using an unpaired Student's two-tailed t test). Statistically significant differences of p<0.05, p<0.01 and p<0.001 are noted with *, ** and ***, respectively.

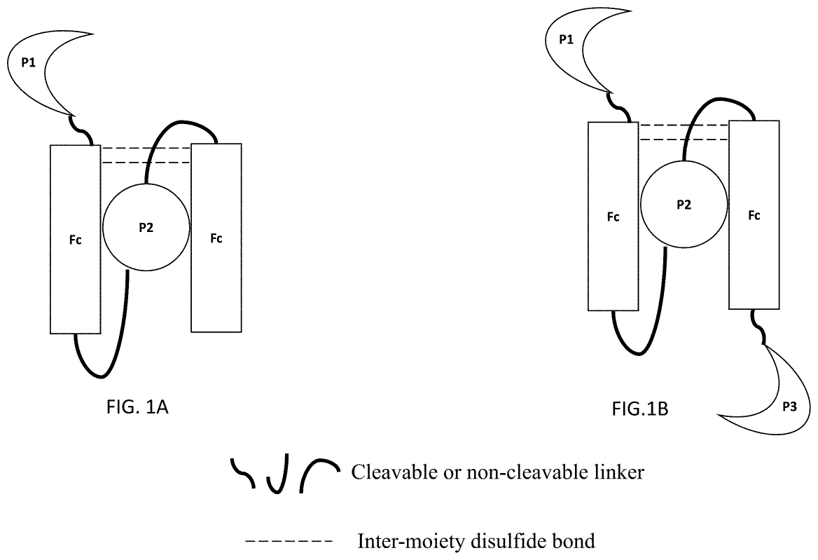

FIGS. 1A and 1B are illustrations of exemplary monomeric cytokine-scFc fusion proteins of the invention with cleavable or non-cleavable linkers to connect the biologically active moiety and Fc region. Linkers are optional in all fusion proteins in the present invention. P1, P2 and P3 is the biologically active moiety, which comprises an antibody, or an antigen-binding fragment, or a cytokine receptor, or a cytokine, or a fusion protein from above-mentioned components. Fc is fragment crystallizable region

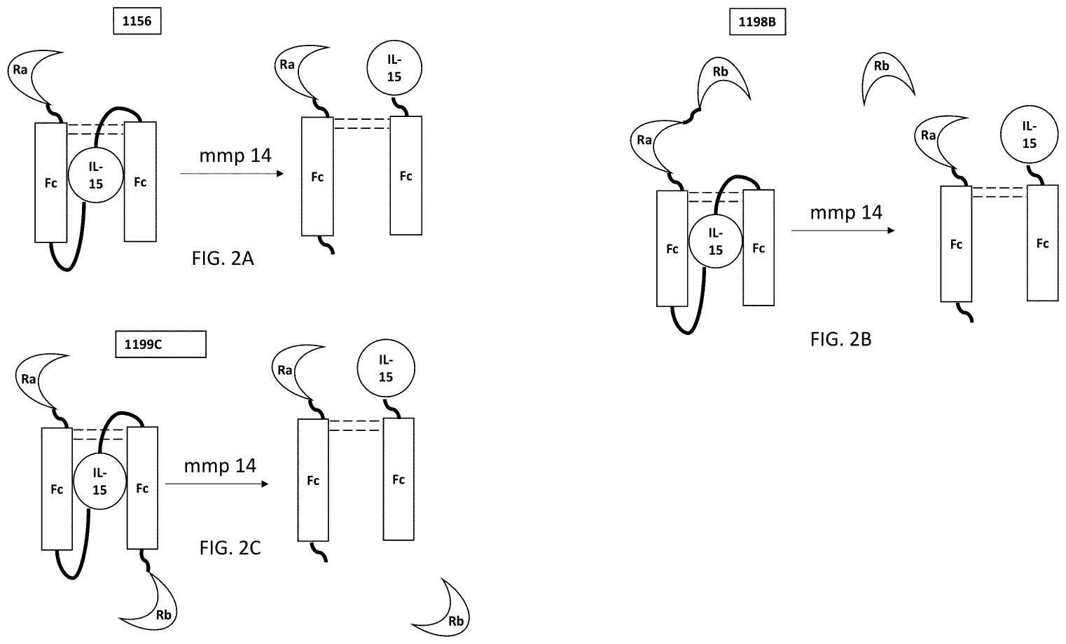

FIGS. 2A to 2C illustrate IL-15-scFc fusion proteins and their cleavage. IL-15Ra-Linker(L)-Fc-L-IL-15-L-Fc (1156) is indicated in FIG. 2A, IL-15Rb-L-IL-15Ra-L-Fc-L-IL-15-L-Fc (1198B) is indicated in FIG. 2B, and IL-15Ra-L-Fc-L-IL-15-L-Fc-L-Rb (1199C) is indicated in FIG. 2C. Cleavage of IL-15-scFc fusion protein with mmp14 enzyme release IL15 from the linkage of one side of Fc and removes inhibitory IL-15 receptor β, which allows most activated complex formation of IL-15 receptor α with IL-15.

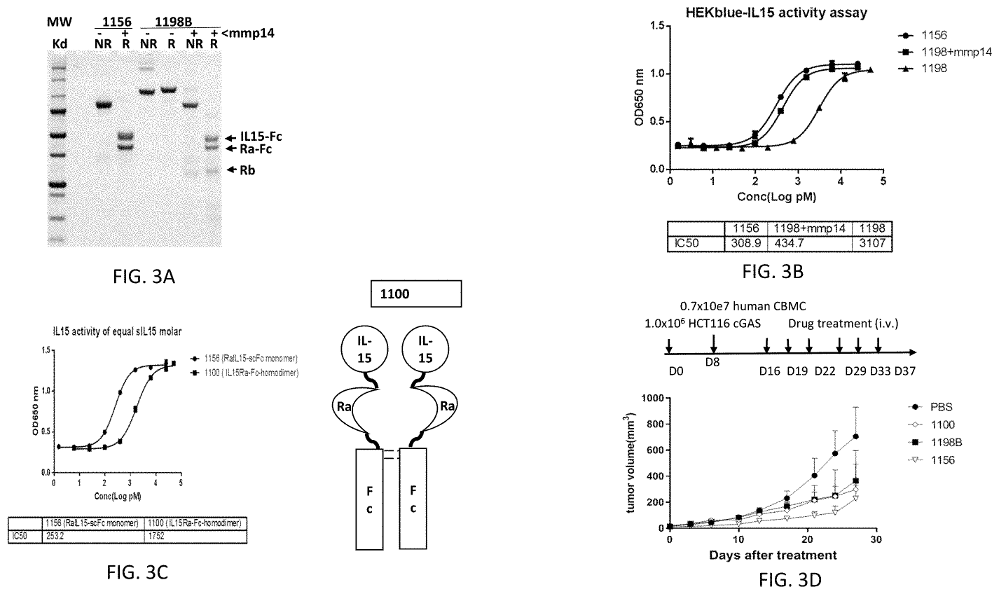

FIG. 3A illustrates pro-drug activation cleavage-mediated by MMP14- digestion of IL-15-scFc fusion proteins in vitro. SDS gel analysis of 1156+/− mmp14 & 1198B+/− mmp14 at reducing (R) and non-reducing (NR) condition. FIG. 3B illustrates IL-15 activity in 1198B increased after cleavage with mmp14, and the activity was measured via HEKblue-IL15 assay. FIGS. 3C and D illustrate that monomeric IL-15-scFc fusion protein exhibited even higher activity than homo-dimeric IL-15Ra-IL-15-Fc fusion protein (1100) in vitro and in vivo, and the activities were measured by HEKblue-IL15 assay and humanized mouse tumor growth model.

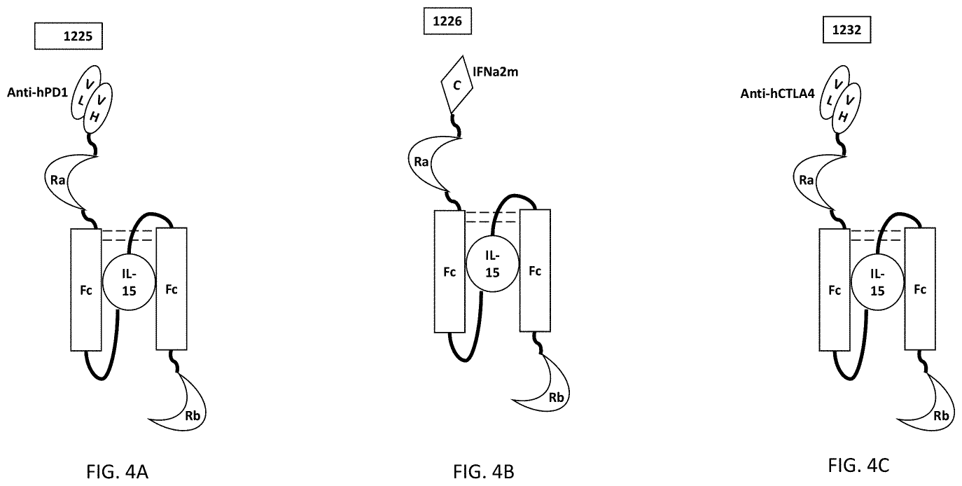

FIGS. 4A, 4B, and 4C illustrate three proteins derived from IL-15-scFc fusion protein (1199C). Their P1 moieties are comprising anti-human PD1 antibody scFv fused to IL-15Rα (1225), IFNα2m- fused to IL-15Ra (1226), and anti-human CTLA4 antibody scFv fused to IL-15Rα (1232), respectively.

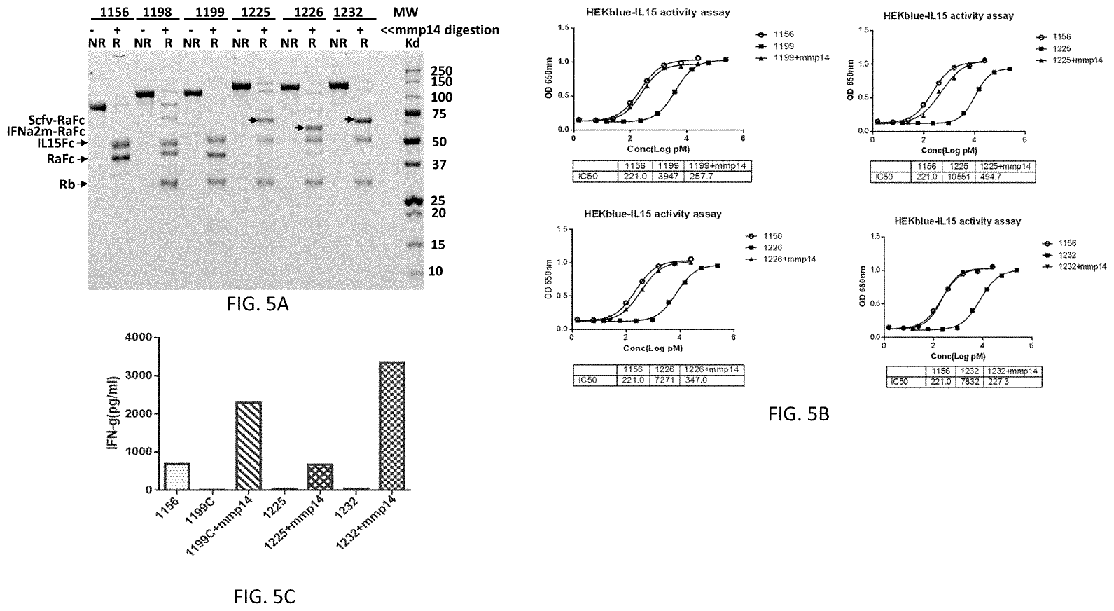

FIG. 5A illustrates the expression and cleavage of different IL-15-scFc fusion proteins, including 1156, 1198, 1199, 1225, 1226, and 1232, analyzed by SDS-PAGE at reducing (R) and non-reducing (NR) condition. FIG. 5B illustrates IL-15 activities from different IL-15-scFc fusion proteins, increased after cleavage with mmp14, and the activities were measured via HEKblue-IL15 assay. FIG. 5C, illustrates IL-15 activities from different IL-15-scFc fusion proteins increased after cleavage with mmp14, and the activities were revealed by IFN gamma production from hu PBMC after stimulated with IL-15-scFc fusion proteins.

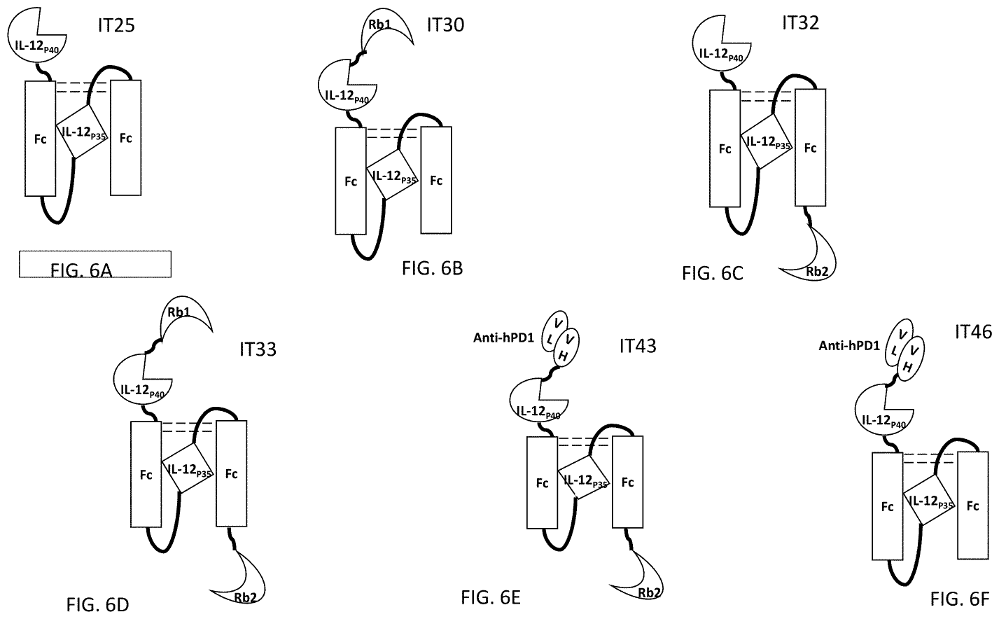

FIGS. 6A to 6F illustrate different monomeric IL-12-scFc fusion proteins fused to antibodies, and/or IL-12 receptor subunits of IL12Rβ1 and IL12Rβ2. IL-12 is a heterodimeric cytokine that includes two subunits: P35 and P40. Interleukin 12 receptor is a type 1 cytokine receptor, consisting of beta 1 and beta 2 subunits that bind to interleukin 12. The positions of the biologically active moieties are interchangeable. FIG. 6A shows IL-12p40-L-Fc-L-IL-12p35-L-Fc (IT25). FIG. 6B shows IL12β1-IL-12p40-L-Fc-L-IL-12p35-L-Fc (IT30). FIG. 6C shows IL-12p40-L-Fc-L-IL-12p35-L-Fc-L-IL12R132 (IT32). FIG. 6D shows IL12Rβ1-IL-12p40-L-Fc-L-IL-12p35-L-Fc-L-IL12Rβ2 (IT33). FIG. 6E shows anti-hPD 1(V_{L}-VH)-L-IL-12p40-L-Fc-L-IL-12p35-L-Fc-L-IL12Rβ2. FIG. 6F shows anti-hPD 1 (V_{L}-VH)-L-IL-12p40-L-Fc-L-IL-12p35-L-Fc.

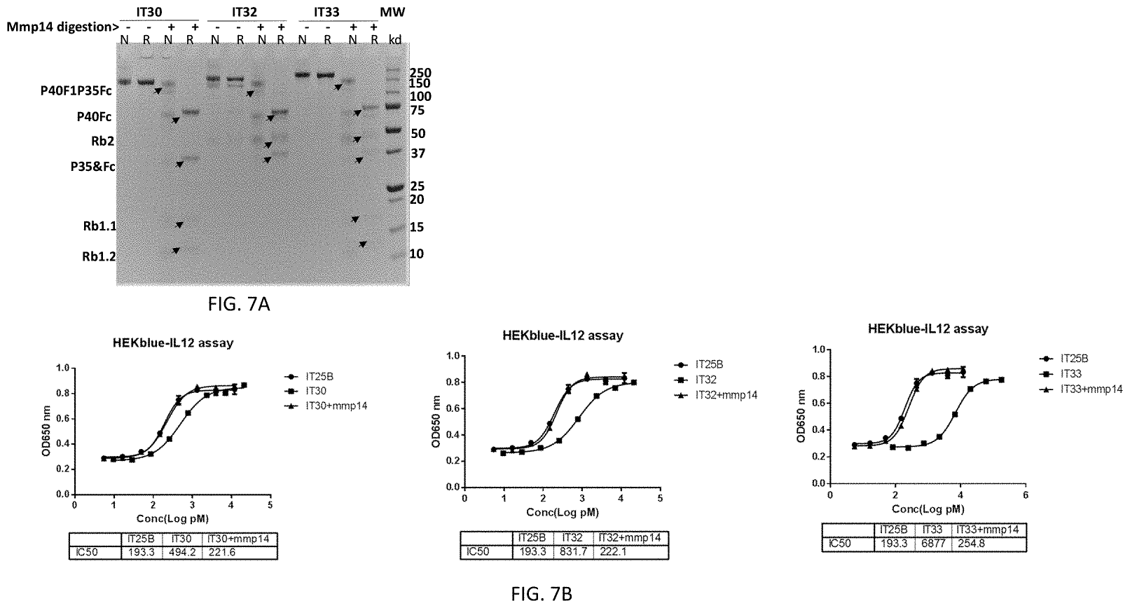

FIG. 7A illustrates pro-drug activation cleavage-mediated by MMP14 digestion of monomeric IL-12-scFc fusion proteins of IT30,32,33. SDS gel analysis of non-cleaved or cleaved proteins at reducing (R) and non-reducing (NR) conditions. Rb1 is cut into two pieces marked as Rβ1.1 and Rβ1.2 from IT30 & 33; Rβ2 is cut out as one fragment from IT32. FIG. 7B illustrates IL-12 activity increased after cleavage with mmp14 measured via HEKblue-IL12 assay.

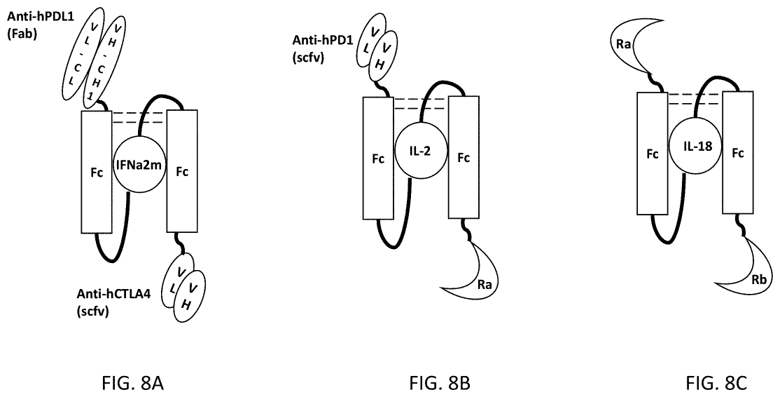

FIGS. 8A, 8B and 8C illustrate other three exemplary monomeric cytokine scFc fusion proteins. FIG. 8A is an Anti-huPDL1(Fab)-L-Fc-IFNα2m-L-Fc-L-anti-huCTLA4 (scfv) fusion protein. FIG. 8B shows an Anti-huPD1(scFv)-L-Fc-L-IL-2-L-Fc-L-Rα fusion protein. FIG. 8C shows a Ra-L-Fc-L-IL-18-L-Fc-L-Rβ fusion protein.

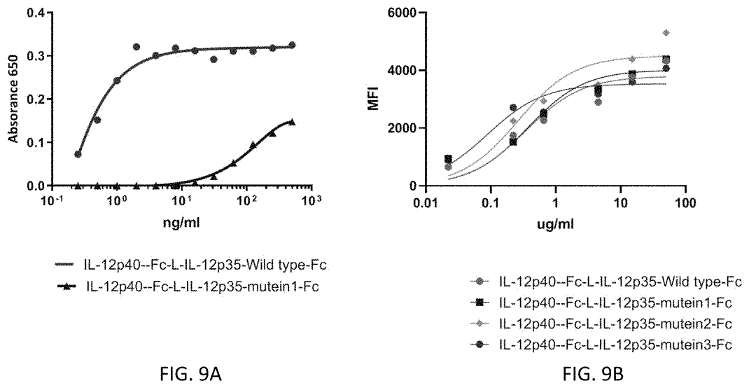

FIGS. 9A and 9B are graphs that illustrate the effect of different constructs. FIG. 9A illustrates that monomeric IL-12p40-Fc-L-IL-12p35-mutein1-Fc fusion protein exhibited lower activities than monomeric IL-12p40-Fc-L-IL-12p35-wild type-Fc (IT25) fusion protein in vitro, and the activities were measured by HEKblue-IL12 assay. 9B illustrates that monomeric IL-12p40-Fc-L-IL-12p35-mutein1/2/3-Fc fusion proteins exhibited comparable binding affinities compared with monomeric IL-12p40-Fc-L-IL-12p35-wild type-Fc (IT25) fusion protein in vitro, and the binding affinities were measured by flow cytometry using recombinant monomeric IL-12p40-Fc-L-IL-12p35-mutein-Fc fusion proteins at a series of concentrations stain on 293 cells expressing IL-12 receptor complex. The IL-12 mutein protein sequences are indicated in SEQ ID NOS: 96-99.

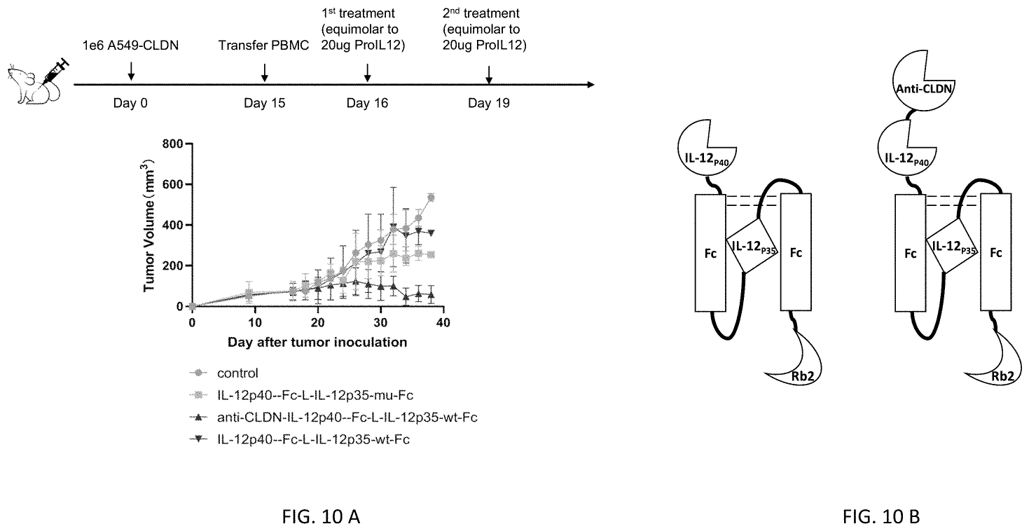

FIGS. 10A and 10B show the results from using different constructs shown in FIG. 10B. FIG. 10A illustrates that target-binding polypeptide (anti-CLDN)-fused IL-12p40-Fc-L-IL-12p35-wild type-Fc inhibits tumor growth in the mouse tumor model. Pro-anti-CLDN-IL-12p40-Fc-L-IL-12p35-wild type-Fc drug at the low concentration significantly inhibits tumor growth in the humanized mouse tumor model, compared with IL-12p40-Fc-L-IL-12p35-wild type-Fc IL-2 treatment. NSG-SGM3 mice were inoculated with A549 expressing CLDN18.2 protein, followed by huPBMC transfer, and the treatment with indicated drugs. The IL-12 mutein protein sequences are indicated in SEQ II) NO:96-99, and the anti-human CLDN18.2 antibody sequences are indicated in SEQ II) NO:92-95. FIG. 10B illustrates illustrate IL-12p40-Fc-L-IL-12p35-wild type/mu-Fc and anti-CLDN-IL-12p40-Fc-L-IL-12p35-wild type-Fc fusion proteins.

SEQ ID NO: 1:
hIL15
NWVNVISDLKKIEDLIQSMHIDATLYTESDVHPSC
KVTAMKCFLLELQVISLESGDASIHDTVENLIILA
NNSLSSNGNVTESGCKECEELEEKNIKEFLQSFVH
IVQMFINTS
SEQ ID NO: 2:
hIgG1Fc (Fcdw)
EPKSSDKTHTCPPCPAPELLGGPSVFLFPPKPKDT
LMISRTPEVTCVVVDVSHEDPEVKFNWYVDGVEVH
NAKTKPREEQYNSTYRVVSVLTVLHQDWLNGKEYK
CKVSNKALPAPIEKTISKAKGQPREPQVYTLPPSR
DELTKNQVSLTCLVKGFYPSDIAVEWESNGQPENN
YKTTPPVLDSDGSFFLYSKLTVDKSRWQQGNVFSC
SVMHEALHNHYTQKSLSLSPGK
SEQ ID NO: 3:
hIgG1Fcmutant (Fcdm):
Fcgamma R binding deficiency.
EPKSSDKTHTCPPCPAPEAAGGPSVFLFPPKPKDT
LMISRTPEVTCVVVDVSHEDPEVKFNWYVDGVEVH
NAKTKPREEQYNSTYRVVSVLTVLHQDWLNGKEYK
CKVSNKALGAPIEKTISKAKGQPREPQVYTLPPSR
DELTKNQVSLTCLVKGFYPSDIAVEWESNGQPENN
YKTTPPVLDSDGSFFLYSKLTVDKSRWQQGNVFSC
SVMHEALHNHYTQKSLSLSPGK
SEQ ID NO: 4:
h15Ra (sushi domain)
ITCPPPMSVEHADIWVKSYSLYSRERYICNSGFKR
KAGTSSLTECVLNKATNVAHWTTPSLKCIRDPALV
HQRPAPP
SEQ ID NO: 5:
h15Rb (27aa~240aa)
AVNGTSQFTCFYNSRANISCVWSQDGALQDTSCQV
HAWPDRRRWNQTCELLPVSQASWACNLILGAPDSQ
KLTTVDIVTLRVLCREGVRWRVMAIQDFKPFENLR
LMAPISLQVVHVETHRCNISWEISQASHYFERHLE
FEARTLSPGHTWEEAPLLTLKQKQEWICLETLTPD
TQYEFQVRVKPLQGEFTTWSPWSQPLAFRTKPAAL
GKDT
SEQ ID NO: 6:
h15Rb (27aa~234aa)
AVNGTSQFTCFYNSRANISCVWSQDGALQDTSCQV
HAWPDRRRWNQTCELLPVSQASWACNLILGAPDSQ
KLTTVDIVTLRVLCREGVRWRVMAIQDFKPFENLR
LMAPISLQVVHVETHRCNISWEISQASHYFERHLE
FEARTLSPGHTWEEAPLLTLKQKQEWICLETLTPD
TQYEFQVRVKPLQGEFTTWSPWSQPLAFRTKPA
SEQ ID NO: 7:
h15Rb (32aa~240aa)
SQFTCFYNSRANISCVWSQDGALQDTSCQVHAWPD
RRRWNQTCELLPVSQASWACNLILGAPDSQKLTTV
DIVTLRVLCREGVRWRVMAIQDFKPFENLRLMAPI
SLQVVHVETHRCNISWEISQASHYFERHLEFEART
LSPGHTWEEAPLLTLKQKQEWICLETLTPDTQYEF
QVRVKPLQGEFTTWSPWSQPLAFRTKPAALGKDT
SEQ ID NO: 8:
h15Rb (32aa~234aa)
SQFTCFYNSRANISCVWSQDGALQDTSCQVHAWPD
RRRWNQTCELLPVSQASWACNLILGAPDSQKLTTV
DIVTLRVLCREGVRWRVMAIQDFKPFENLRLMAPI
SLQVVHVETHRCNISWEISQASHYFERHLEFEART
LSPGHTWEEAPLLTLKQKQEWICLETLTPDTQYEF
QVRVKPLQGEFTTWSPWSQPLAFRTKPA
SEQ ID NO: 9:
h15Rb (27aa~125aa)
AVNGTSQFTCFYNSRANISCVWSQDGALQDTSCQV
HAWPDRRRWNQTCELLPVSQASWACNLILGAPDSQ
KLTTVDIVTLRVLCREGVRWRVMAIQDFK
SEQ ID NO: 10:
h15Rb (27aa~128aa)
AVNGTSQFTCFYNSRANISCVWSQDGALQDTSCQV
HAWPDRRRWNQTCELLPVSQASWACNLILGAPDSQ
KLTTVDIVTLRVLCREGVRWRVMAIQDFKPFE
SEQ ID NO: 11:
h15Rb (27aa~137aa)
AVNGTSQFTCFYNSRANISCVWSQDGALQDTSCQV
HAWPDRRRWNQTCELLPVSQASWACNLILGAPDSQ
KLTTVDIVTLRVLCREGVRWRVMAIQDFKPFENLR
LMAPIS
SEQ ID NO: 12:
h15Rb (32aa~137aa)
SQFTCFYNSRANISCVWSQDGALQDTSCQVHAWPD
RRRWNQTCELLPVSQASWACNLILGAPDSQKLTTV
DIVTLRVLCREGVRWRVMAIQDFKPFENLRLMAPI
S

Non-cleavable linkers.

SEQ ID NO: 100:
(GGS)n, n = 1~10;
SEQ ID NO: 13:
(GGGS)n, n = 1~10;
SEQ ID NO: 14:
(GGGGS)n, n = 1~10;
SEQ ID NO: 15:
(SGGGS)n, n = 1~10;
SEQ ID NO: 16:
SGGGSGGGGS GGGGSGGGGS GGGSLQ

Cleavable mmp substrate sequences

SEQ ID NO: 17:
-SGRSPAIFTA-;
SEQ ID NO: 18:
-SGRSENIRTA-
SEQ ID NO: 19:
-SGARYRWLTA-;
SEQ ID NO: 20:
-SGRSYAILTA-;
SEQ ID NO: 21:
-SGRAMHMYTA-
SEQ ID NO: 22:
-SPVGLIG-;
SEQ ID NO: 23:
-SGRIGFLRTA-;
SEQ ID NO: 24:
-SGAIGFLRTA-;
SEQ ID NO: 25:
-SGAAMHMYTA-
SEQ ID NO: 26:
-SGASENIRTA-;
SEQ ID NO: 27:
-SGRPENIRTA-;
SEQ ID NO: 28:
-SGAPENIRTA-;
SEQ ID NO: 29:
-SGLISHSITA-;
SEQ ID NO: 30:
-SGNLRSKLTA-;
SEQ ID NO: 31:
SGVFSIPLTA;
SEQ ID NO: 32:
SGIKYHSLTA;

Cleavable MMP substrate sequences-long linker, composed of following two short substrate sequences as examples, but not excluding others combination and orders:

SEQ ID NO: 33:
-SGRSPAIFTA-L-SGRSENIRTA-
SEQ ID NO: 34:
-SGRSENIRTA-L-SGRSPAIFTA-
SEQ ID NO: 35:
-SGARYRWLTA-L-SGRSENIRTA-
SEQ ID NO: 36:
-SGRSENIRTA-L-SGARYRWLTA-
SEQ ID NO: 37:
-SGRAMHMYTA-L-SGRSENIRTA-
SEQ ID NO: 38:
-SGRAMHMYTA-L-SGRAMHMYTA-
SEQ ID NO: 39:
-SGRSPAIFTA-L-SGARYRWLTA-
SEQ ID NO: 40:
-SGARYRWLTA-L-SGRSPAIFTA-
SEQ ID NO: 41:
-SGRSPAIFTA-L-SGRSYAILTA-
SEQ ID NO: 42:
-SGRSYAILTA-L-SGRSPAIFTA-
SEQ ID NO: 43:
-SGRSPAIFTA-L-SGRAMHMYTA-
SEQ ID NO: 44:
-SGRAMHMYTA-L-SGRSPAIFTA-
SEQ ID NO: 45:
-SGARYRWLTA-L-SGRSYAILTA-
SEQ ID NO: 46:
-SGRSYAILTA-L-SGARYRWLTA-
SEQ ID NO: 47:
-SGARYRWLTA-L-SGRAMHMYTA-
SEQ ID NO: 48:
-SGRAMHMYTA-L-SGARYRWLT-
SEQ ID NO: 49:
-SGRSYAILTA-L-SGRAMHMYTA-
SEQ ID NO: 50:
-SGRAMHMYTA-L-SGRSYAILTA-
SEQ ID NO: 51:
CH₁:
ASTKGPSVFPLAPSSKSTSGGTAALGCLVKDYFP
EPVTVSWNSGALTSGVHTFPAVLQSSGLYSLSSV
VTVPSSSLGTQTYICNVNHKPSNTKVDKRVEPKSC
SEQ ID NO: 52:
C_{L}
RTVAAPSVFIFPPSDEQLKSGTASVVCLLNNFYPREAK
VQWKVDNALQSGNSQESVTEQDSKDSTYSLSSTLTLSK
ADYEKHKVYACEVTHQGLSSPVTKSFNRGEC
SEQ ID NO: 53:
IgkL sequence
METDTLLLWVLLLWVPGSTGD
SEQ ID NO: 54:
human IL-12 P40 subunit (no signal peptide)
WELKKDVYVVELDWYPDAPGEMVVLTCDTPEEDGITWTLD
QSSEVLGSGKTLTIQVKEFGDAGQYTCHKGGEVLSHSLLL
LHKKEDGIWSTDILKDQKEPKNKTFLRCEAKNYSGRFTCW
WLTTISTDLTFSVKSSRGSSDPQGVTCGAATLSAERVRGD
NKEYEYSVECQEDSACPAAEESLPIEVMVDAVHKLKYENY
TSSFFIRDIIKPDPPKNLQLKPLKNSRQVEVSWEYPDTWS
TPHSYFSLTFCVQVQGKSKREKKDRVFTDKTSATVICRKN
ASISVRAQDRYYSSSWSEWASVPCS
SEQ ID NO: 55:
human IL-12 P35 subunit (no signal peptide)
RNLPVATPDPGMFPCLHHSQNLLRAVSNMLQKARQTLEFY
PCTSEEIDHEDITKDKTSTVEACLPLELTKNESCLNSRET
SFITNGSCLASRKTSFMMALCLSSIYEDLKMYQVEFKTMN
AKLLMDPKRQIFLDQNMLAVIDELMQALNFNSETVPQKSS
LEEPDFYKTKIKLCILLHAFRIRAVTIDRVMSYLNAS
SEQ ID NO: 56:
mutant human Fc-hole (Fc-h)
DKTHTCPPCP APEAAGGPSV FLFPPKPKDT
LMISRTPEVT CVVVDVSHED PEVKFNWYVD
GVEVHNAKTK PREEQYNSTY RVVSVLTVLH
QDWLNGKEYK CKVSNKALPA PIEKTISKAK
GQPREPQVCT LPPSRDELTK NQVSLSCAVK
GFYPSDIAVE WESNGQPENN YKTTPPVLD
SDGSFFLVSKL TVDKSRWQQG NVFSCSVMHE
ALHNHYTQKS LSLSPGK
SEQ ID NO: 57:
mutant human Fc-knob (Fc-k)
DKTHTCPPCPAPEAAGGPSVFLFPPKPKDTLMISRTPEVT
CWVDVSHEDPEVKFNWYVDGVEVHNAKTKPREEQYNSTYR
VVSVLTVLHQDWLNGKEYKCKVSNKALGAPIEKTISKAKG
QPREPQVCTLPPSRDELTKNQVSLSCAVKGFYPSDIAVEW
ESNGQPENNYKTTPPVLDSDGSFKLVSKLTVDKSRWQQGN
VFSCSVMHEALHNHYTQKSLSLSPGKHV
SEQ ID NO: 58:
human IL-12Rβ1
MEPLVTWVVPLLFLFAASRQGAACRTSECCFQDPPYPDAD
SGSASGPRDLRCYRISSDRYECSWQYEGPTAGVSHFLRCC
LSSGRCCYFAAGSATRLQFSDQAGVSVLYTVTLWVESWAR
NQTEKSPEVTLQLYNSVKYEPPLGDIKVSKLAGQLRMEWE
TPDNQVGAEVQFRHRTPSSPWKLGDCGPQDDDTESCLCPL
EMNVAQEFQLRRRRLGSQGSSWSKWSSPVCVPPEN
SEQ ID NO: 59:
human IL-12Rβ2
MAHTFRGCSLAFMFIITWLLIKAKIDACKRGDVTVKPSHV
ILLGSTVNITCSLKPRQGCFHYSRRNKLILYKFDRRINFH
HGHSLNSQVTGLPLGTTLFVCKLACINSDEIQICGAEIFV
GVAPEQPQNLSCIQKGEQGTVACTWERGRDTHLYTEYTLQ
LSGPKNLTWQKQCKDIYCDYLDFGINLTPESPESNFTAKV
TAVNSLGSSSSLPSTFTFLDIVRPLPPWDIRIKFQKASVS
RCTLYWRDEGLVLLNRLRYRPSNSRLWNMVNVTKAKGRHD
LLDLKPFTEYEFQISSKLHLYKGSWSDWSESLRAQTPEE
SEQ ID NO: 60:
28.1: VH(a-hPD11)-L-CH1-Fc-L(*)-
hIFNa2m-L-Fc (1213)
METDTLLLWVLLLWVPGSTGDQVQLVQSGAEVKKPGSSVK
VSCKTSGDTFSTYAISWVRQAPGQGLEWMGGIIPIFGKAH
YAQKFQGRVTITADESTSTAYMELSSLRSEDTAVYFCARK
FHFVSGSPFGMDVWGQGTTVTVSSASTKGPSVFPLAPSSK
STSGGTAALGCLVKDYFPEPVTVSWNSGALTSGVHTFPAV
LQSSGLYSLSSVVTVPSSSLGTQTYICNVNHKPSNTKVDK
RVEPKSCEPKSSDKTHTCPPCPAPELLGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
PAPIEKTISKAKGQPREPQVYTLPPSRDELTKNQVSLTCL
VKGFYPSDIAVEWESNGQPENNYKTTPPVLDSDGSFFLYS
KLTVDKSRWQQGNVFSCSVMHEALHNHYTQKSLSLSPGKG
GSSGARYRWLTAGGSGRSENIRTAGGSCDLPQTHSLGSRR
TLMLLAQMRRISLFSCLKDRHDFGFPQEEFGNQFQKAETI
PVLHEMIQQIFNLFSTKDSSAAWDETLLDKFYTELYQQLN
DLEACVIQGVGVTETPLMKEDSILAVRKYFRRITLYLKEK
KYSPCAWEVVRAEIMRSFSLSTNLQESLRSKESGGGSEPK
SSDKTHTCPPCPAPELLGGPSVFLFPPKPKDTLMISRTPE
VTCVVVDVSHEDPEVKFNWYVDGVEVHNAKTKPREEQYNS
TYRVVSVLTVLHQDWLNGKEYKCKVSNKALPAPIEKTISK
AKGQPREPQVYTLPPSRDELTKNQVSLTCLVKGFYPSDIA
VEWESNGQPENNYKTTPPVLDSDGSFFLYSKLTVDKSRWQ
QGNVFSCSVMHEALHNHYTQKSLSLSPGK
SEQ ID NO: 61:
28.2: VL (a-hPD11)-CL (1143)
METDTLLLWVLLLWVPGSTGDEIVLTQSPATLSLSPGERA
TLSCRASQSVSSYLAWYQQKPGQAPRLLIYDASNRATGIP
ARFSGSGSGTDFTLTISSLEPEDFAVYYCQQRSNWPTFGQ
GTKVEIKRTVAAPSVFIFPPSDEQLKSGTASVVCLLNNFY
PREAKVQWKVDNALQSGNSQESVTEQDSKDSTYSLSSTLT
LSKADYEKHKVYACEVTHQGLSSPVTKSFNRGEC
SEQ ID NO: 62:
29.1: VH(a-hPD11)-L-CH1-Fc-L-
hIFNa2m-L(*)-Fc (1221)
METDTLLLWVLLLWVPGSTGDQVQLVQSGAEVKKPGSSVK
VSCKTSGDTFSTYAISWVRQAPGQGLEWMGGIIPIFGKAH
YAQKFQGRVTITADESTSTAYMELSSLRSEDTAVYFCARK
FHFVSGSPFGMDVWGQGTTVTVSSASTKGPSVFPLAPSSK
STSGGTAALGCLVKDYFPEPVTVSWNSGALTSGVHTFPAV
LQSSGLYSLSSVVTVPSSSLGTQTYICNVNHKPSNTKVDK
RVEPKSCEPKSSDKTHTCPPCPAPELLGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
PAPIEKTISKAKGQPREPQVYTLPPSRDELTKNQVSLTCL
VKGFYPSDIAVEWESNGQPENNYKTTPPVLDSDGSFFLYS
KLTVDKSRWQQGNVFSCSVMHEALHNHYTQKSLSLSPGKG
GSSGAGGSGRSGGSCDLPQTHSLGSRRTLMLLAQMRRISL
FSCLKDRHDFGFPQEEFGNQFQKAETIPVLHEMIQQIFNL
FSTKDSSAAWDETLLDKFYTELYQQLNDLEACVIQGVGVT
ETPLMKEDSILAVRKYFRRITLYLKEKKYSPCAWEVVRAE
IMRSFSLSTNLQESLRSKESGGARYRWLGRSENIRTGSEP
KSSDKTHTCPPCPAPELLGGPSVFLFPPKPKDTLMISRTP
EVTCVVVDVSHEDPEVKFNWYVDGVEVHNAKTKPREEQYN
STYRVVSVLTVLHQDWLNGKEYKCKVSNKALPAPIEKTIS
KAKGQPREPQVYTLPPSRDELTKNQVSLTCLVKGFYPSDI
AVEWESNGQPENNYKTTPPVLDSDGSFFLYSKLTVDKSRW
QQGNVFSCSVMHEALHNHYTQKSLSLSPGK
SEQ ID NO: 63:
VL (a-hPD11)-CL (1143)
METDTLLLWVLLLWVPGSTGDEIVLTQSPATLSLSPGERA
TLSCRASQSVSSYLAWYQQKPGQAPRLLIYDASNRATGIP
ARFSGSGSGTDFTLTISSLEPEDFAVYYCQQRSNWPTFGQ
GTKVEIKRTVAAPSVFIFPPSDEQLKSGTASVVCLLNNFY
PREAKVQWKVDNALQSGNSQESVTEQDSKDSTYSLSSTLT
LSKADYEKHKVYACEVTHQGLSSPVTKSFNRGEC
SEQ ID NO: 64:
30.1: VH(a-PDL1)-CH1-L-Fc-L (*)-
hIFNa2m-L-Fc-L-scFV (a-
CTLA4) (1222)
METDTLLLWVLLLWVPGSTGDQVQLVQSGAEVKKPGSSVK
VSCKTSGDTFSTYAISWVRQAPGQGLEWMGGIIPIFGKAH
YAQKFQGRVTITADESTSTAYMELSSLRSEDTAVYFCARK
FHFVSGSPFGMDVWGQGTTVTVSSASTKGPSVFPLAPSSK
STSGGTAALGCLVKDYFPEPVTVSWNSGALTSGVHTFPAV
LQSSGLYSLSSVVTVPSSSLGTQTYICNVNHKPSNTKVDK
RVEPKSCEPKSSDKTHTCPPCPAPELLGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
PAPIEKTISKAKGQPREPQVYTLPPSRDELTKNQVSLTCL
VKGFYPSDIAVEWESNGQPENNYKTTPPVLDSDGSFFLYS
KLTVDKSRWQQGNVFSCSVMHEALHNHYTQKSLSLSPGKG
GSSGARYRWLTAGGSGRSENIRTAGGSCDLPQTHSLGSRR
TLMLLAQMRRISLFSCLKDRHDFGFPQEEFGNQFQKAETI
PVLHEMIQQIFNLFSTKDSSAAWDETLLDKFYTELYQQLN
DLEACVIQGVGVTETPLMKEDSILAVRKYFRRITLYLKEK
KYSPCAWEVVRAEIMRSFSLSTNLQESLRSKESGGGSEPK
SSDKTHTCPPCPAPELLGGPSVFLFPPKPKDTLMISRTPE
VTCVVVDVSHEDPEVKFNWYVDGVEVHNAKTKPREEQYNS
TYRVVSVLTVLHQDWLNGKEYKCKVSNKALPAPIEKTISK
AKGQPREPQVYTLPPSRDELTKNQVSLTCLVKGFYPSDIA
VEWESNGQPENNYKTTPPVLDSDGSFFLYSKLTVDKSRWQ
QGNVFSCSVMHEALHNHYTQKSLSLSPGKSRPHSRGGGSG
GGSTGRSEIVLTQSPGTLSLSPGERATLSCRASQSVGSSY
LAWYQQKPGQAPRLLIYGAFSRATGIPDRFSGSGSGTDFT
LTISRLEPEDFAVYYCQQYGSSPWTFGQGTKVEIKGGGGS
GGGGSGGGGSQVQLVESGGGVVQPGRSLRLSCAASGFTFS
SYTMHWVRQAPGKGLEWVTFISYDGNNKYYADSVKGRFTI
SRDNSKNTLYLQMNSLRAEDTAIYYCARTGWLGPFDYWGQ
GTLVTVSS
SEQ ID NO: 65:
28.2: VL (a-hPD11)-CL (1143)
METDTLLLWVLLLWVPGSTGDEIVLTQSPATLSLSPGERA
TLSCRASQSVSSYLAWYQQKPGQAPRLLIYDASNRATGIP
ARFSGSGSGTDFTLTISSLEPEDFAVYYCQQRSNWPTFGQ
GTKVEIKRTVAAPSVFIFPPSDEQLKSGTASVVCLLNNFY
PREAKVQWKVDNALQSGNSQESVTEQDSKDSTYSLSSTLT
LSKADYEKHKVYACEVTHQGLSSPVTKSFNRGEC
SEQ ID NO: 66:
31.1: VH-(a-PDL1)-L-CH1-Fc-L-hIFNa2m-
L(*)-Fc-L- scFV (a-
hCTLA4) (1264)
METDTLLLWVLLLWVPGSTGDQVQLVQSGAEVKKPGSSVK
VSCKTSGDTFSTYAISWVRQAPGQGLEWMGGIIPIFGKAH
YAQKFQGRVTITADESTSTAYMELSSLRSEDTAVYFCARK
FHFVSGSPFGMDVWGQGTTVTVSSASTKGPSVFPLAPSSK
STSGGTAALGCLVKDYFPEPVTVSWNSGALTSGVHTFPAV
LQSSGLYSLSSVVTVPSSSLGTQTYICNVNHKPSNTKVDK
RVEPKSCEPKSSDKTHTCPPCPAPELLGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
PAPIEKTISKAKGQPREPQVYTLPPSRDELTKNQVSLTCL
VKGFYPSDIAVEWESNGQPENNYKTTPPVLDSDGSFFLYS
KLTVDKSRWQQGNVFSCSVMHEALHNHYTQKSLSLSPGKG
GSSGAGGSGRSGGSCDLPQTHSLGSRRTLMLLAQMRRISL
FSCLKDRHDFGFPQEEFGNQFQKAETIPVLHEMIQQIFNL
FSTKDSSAAWDETLLDKFYTELYQQLNDLEACVIQGVGVT
ETPLMKEDSILAVRKYFRRITLYLKEKKYSPCAWEVVRAE
IMRSFSLSTNLQESLRSKESGGARYRWLGRSENIRTGSEP
KSSDKTHTCPPCPAPELLGGPSVFLFPPKPKDTLMISRTP
EVTCVVVDVSHEDPEVKFNWYVDGVEVHNAKTKPREEQYN
STYRVVSVLTVLHQDWLNGKEYKCKVSNKALPAPIEKTIS
KAKGQPREPQVYTLPPSRDELTKNQVSLTCLVKGFYPSDI
AVEWESNGQPENNYKTTPPVLDSDGSFFLYSKLTVDKSRW
QQGNVFSCSVMHEALHNHYTQKSLSLSPGKSRPHSRGGGS
GGGSTGRSEIVLTQSPGTLSLSPGERATLSCRASQSVGSS
YLAWYQQKPGQAPRLLIYGAFSRATGIPDRFSGSGSGTDF
TLTISRLEPEDFAVYYCQQYGSSPWTFGQGTKVEIKGGGG
SGGGGSGGGGSQVQLVESGGGVVQPGRSLRLSCAASGFTF
SSYTMHWVRQAPGKGLEWVTFISYDGNNKYYADSVKGRFT
ISRDNSKNTLYLQMNSLRAEDTAIYYCARTGWLGPFDYWG
QGTLVTVSS
SEQ ID NO: 67:
32.1: VH (a-hPD11)-L-CH1-Fc-L-VH
(a-CTLA4)-L (*)-hIFNa2m-
L-Fc-L-VL (a-CTLA4) (1269)
METDTLLLWVLLLWVPGSTGDQVQLVQSGAEVKKPGSSVK
VSCKTSGDTFSTYAISWVRQAPGQGLEWMGGIIPIFGKAH
YAQKFQGRVTITADESTSTAYMELSSLRSEDTAVYFCARK
FHFVSGSPFGMDVWGQGTTVTVSSASTKGPSVFPLAPSSK
STSGGTAALGCLVKDYFPEPVTVSWNSGALTSGVHTFPAV
LQSSGLYSLSSVVTVPSSSLGTQTYICNVNHKPSNTKVDK
RVEPKSCEPKSSDKTHTCPPCPAPELLGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
PAPIEKTISKAKGQPREPQVYTLPPSRDELTKNQVSLTCL
VKGFYPSDIAVEWESNGQPENNYKTTPPVLDSDGSFFLYS
KLTVDKSRWQQGNVFSCSVMHEALHNHYTQKSLSLSPGKG
GSGTGGFESQVQLVESGGGVVQPGRSLRLSCAASGFTFSS
YTMHWVRQAPGKGLEWVTFISYDGNNKYYADSVKGRFTIS
RDNSKNTLYLQMNSLRAEDTAIYYCARTGWLGPFDYWGQG
TLVTVSSGARYRWLTAGGSGRSENIRTAGGSCDLPQTHSL
GSRRTLMLLAQMRRISLFSCLKDRHDFGFPQEEFGNQFQK
AETIPVLHEMIQQIFNLFSTKDSSAAWDETLLDKFYTELY
QQLNDLEACVIQGVGVTETPLMKEDSILAVRKYFRRITLY
LKEKKYSPCAWEVVRAEIMRSFSLSTNLQESLRSKESGGG
SEPKSSDKTHTCPPCPAPELLGGPSVFLFPPKPKDTLMIS
RTPEVTCVVVDVSHEDPEVKFNWYVDGVEVHNAKTKPREE
QYNSTYRVVSVLTVLHQDWLNGKEYKCKVSNKALPAPIEK
TISKAKGQPREPQVYTLPPSRDELTKNQVSLTCLVKGFYP
SDIAVEWESNGQPENNYKTTPPVLDSDGSFFLYSKLTVDK
SRWQQGNVFSCSVMHEALHNHYTQKSLSLSPGKSRPHSRG
GGSGGGSTGRSEIVLTQSPGTLSLSPGERATLSCRASQSV
GSSYLAWYQQKPGQAPRLLIYGAFSRATGIPDRFSGSGSG
TDFTLTISRLEPEDFAVYYCQQYGSSPWTFGQGTKVEIK
SEQ ID NO: 68:
28.2: VL (a-hPD11)-CL (1143)
METDTLLLWVLLLWVPGSTGDEIVLTQSPATLSLSPGERA
TLSCRASQSVSSYLAWYQQKPGQAPRLLIYDASNRATGIP
ARFSGSGSGTDFTLTISSLEPEDFAVYYCQQRSNWPTFGQ
GTKVEIKRTVAAPSVFIFPPSDEQLKSGTASVVCLLNNFY
PREAKVQWKVDNALQSGNSQESVTEQDSKDSTYSLSSTLT
LSKADYEKHKVYACEVTHQGLSSPVTKSFNRGEC
SEQ ID NO: 69:
Ra (sushi)-L-Fc-L (*)-IL15-L-Fc (1156)
METDTLLLWVLLLWVPGSTGDITCPPPMSVEHADIWVKSY
SLYSRERYICNSGFKRKAGTSSLTECVLNKATNVAHWTTP
SLKCIRDPALVHQRPAPPGGGGSEPKSSDKTHTCPPCPAP
ELLGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALPAPIEKTISKAKGQPREPQVYTLP
PSRDELTKNQVSLTCLVKGFYPSDIAVEWESNGQPENNYK
TTPPVLDSDGSFFLYSKLTVDKSRWQQGNVFSCSVMHEAL
HNHYTQKSLSLSPGKGGSSGARYRWLTAGGSGRSENIRTA
GGSNWVNVISDLKKIEDLIQSMHIDATLYTESDVHPSCKV
TAMKCFLLELQVISLESGDASIHDTVENLIILANNSLSSN
GNVTESGCKECEELEEKNIKEFLQSFVHIVQMFINTSSGG
GSEPKSSDKTHTCPPCPAPELLGGPSVFLFPPKPKDTLMI
SRTPEVTCVVVDVSHEDPEVKFNWYVDGVEVHNAKTKPRE
EQYNSTYRVVSVLTVLHQDWLNGKEYKCKVSNKALPAPIE
KTISKAKGQPREPQVYTLPPSRDELTKNQVSLTCLVKGFY
PSDIAVEWESNGQPENNYKTTPPVLDSDGSFFLYSKLTVD
KSRWQQGNVFSCSVMHEALHNHYTQKSLSLSPGK
SEQ ID NO: 70:
Ra (sushi)-L-Fc-L (*)-IL15-L-Fc-L-
IFNam2 (1224)
METDTLLLWVLLLWVPGSTGDITCPPPMSVEHADIWVKSY
SLYSRERYICNSGFKRKAGTSSLTECVLNKATNVAHWTTP
SLKCIRDPALVHQRPAPPGGGGSEPKSSDKTHTCPPCPAP
ELLGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALPAPIEKTISKAKGQPREPQVYTLP
PSRDELTKNQVSLTCLVKGFYPSDIAVEWESNGQPENNYK
TTPPVLDSDGSFFLYSKLTVDKSRWQQGNVFSCSVMHEAL
HNHYTQKSLSLSPGKGGSSGARYRWLTAGGSGRSENIRTA
GGSNWVNVISDLKKIEDLIQSMHIDATLYTESDVHPSCKV
TAMKCFLLELQVISLESGDASIHDTVENLIILANNSLSSN
GNVTESGCKECEELEEKNIKEFLQSFVHIVQMFINTSSGG
GSEPKSSDKTHTCPPCPAPELLGGPSVFLFPPKPKDTLMI
SRTPEVTCVVVDVSHEDPEVKFNWYVDGVEVHNAKTKPRE
EQYNSTYRVVSVLTVLHQDWLNGKEYKCKVSNKALPAPIE
KTISKAKGQPREPQVYTLPPSRDELTKNQVSLTCLVKGFY
PSDIAVEWESNGQPENNYKTTPPVLDSDGSFFLYSKLTVD
KSRWQQGNVFSCSVMHEALHNHYTQKSLSLSPGKSRPHSR
GGGSGGGSTGRSCDLPQTHSLGSRRTLMLLAQMRRISLFS
CLKDRHDFGFPQEEFGNQFQKAETIPVLHEMIQQIFNLFS
TKDSSAAWDETLLDKFYTELYQQLNDLEACVIQGVGVTET
PLMKEDSILAVRKYFRRITLYLKEKKYSPCAWEVVRAEIM
RSFSLSTNLQESLRSKE
SEQ ID NO: 71:
scFV (a-hPD1)-L-Ra (sushi)-L-Fc-L (*)-
IL15-L-F2c (1255)
METDTLLLWVLLLWVPGSTGDEIVLTQSPATLSLSPGERA
TLSCRASKGVSTSGYSYLHWYQQKPGQAPRLLIYLASYLE
SGVPARFSGSGSGTDFTLTISSLEPEDFAVYYCQHSRDLP
LTFGGGTKVEIKRTGGGGSGGGGSGGGGSQVQLVQSGVEV
KKPGASVKVSCKASGYTFTNYYMYWVRQAPGQGLEWMGGI
NPSNGGTNFNEKFKNRVTLTTDSSTTTAYMELKSLQFDDT
AVYYCARRDYRFDMGFDYWGQGTTVTVSSGGSGGITCPPP
MSVEHADIWVKSYSLYSRERYICNSGFKRKAGTSSLTECV
LNKATNVAHWTTPSLKCIRDPALVHQRPAPPGGGGSEPKS
SDKTHTCPPCPAPELLGGPSVFLFPPKPKDTLMISRTPEV
TCVVVDVSHEDPEVKFNWYVDGVEVHNAKTKPREEQYNST
YRVVSVLTVLHQDWLNGKEYKCKVSNKALPAPIEKTISKA
KGQPREPQVYTLPPSRDELTKNQVSLTCLVKGFYPSDIAV
EWESNGQPENNYKTTPPVLDSDGSFFLYSKLTVDKSRWQQ
GNVFSCSVMHEALHNHYTQKSLSLSPGKGGSSGARYRWLT
AGGSGRSENIRTAGGSNWVNVISDLKKIEDLIQSMHIDAT
LYTESDVHPSCKVTAMKCFLLELQVISLESGDASIHDTVE
NLIILANNSLSSNGNVTESGCKECEELEEKNIKEFLQSFV
HIVQMFINTSSGGGSEPKSSDKTHTCPPCPAPELLGGPSV
FLFPPKPKDTLMISRTPEVTCVVVDVSHEDPEVKFNWYVD
GVEVHNAKTKPREEQYNSTYRVVSVLTVLHQDWLNGKEYK
CKVSNKALPAPIEKTISKAKGQPREPQVYTLPPSRDELTK
NQVSLTCLVKGFYPSDIAVEWESNGQPENNYKTTPPVLDS
DGSFFLYSKLTVDKSRWQQGNVFSCSVMHEALHNHYTQKS
LSLSPGK
SEQ ID NO: 72:
hIFNa2m-L-Ra (sushi)-L-Fc-L (*)-IL15-L-Fc
(1256)
METDTLLLWVLLLWVPGSTGDCDLPQTHSLGSRRTLMLLA
QMRRISLFSCLKDRHDFGFPQEEFGNQFQKAETIPVLHEM
IQQIFNLFSTKDSSAAWDETLLDKFYTELYQQLNDLEACV
IQGVGVTETPLMKEDSILAVRKYFRRITLYLKEKKYSPCA
WEVVRAEIMRSFSLSTNLQESLRSKESGGGSITCPPPMSV
EHADIWVKSYSLYSRERYICNSGFKRKAGTSSLTECVLNK
ATNVAHWTTPSLKCIRDPALVHQRPAPPGGGGSEPKSSDK
THTCPPCPAPELLGGPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSRDELTKNQVSLTCLVKGFYPSDIAVEWE
SNGQPENNYKTTPPVLDSDGSFFLYSKLTVDKSRWQQGNV
FSCSVMHEALHNHYTQKSLSLSPGKGGSSGARYRWLTAGG
SGRSENIRTAGGSNWVNVISDLKKIEDLIQSMHIDATLYT
ESDVHPSCKVTAMKCFLLELQVISLESGDASIHDTVENLI
ILANNSLSSNGNVTESGCKECEELEEKNIKEFLQSFVHIV
QMFINTSSGGGSEPKSSDKTHTCPPCPAPELLGGPSVFLF
PPKPKDTLMISRTPEVTCVVVDVSHEDPEVKFNWYVDGVE
VHNAKTKPREEQYNSTYRVVSVLTVLHQDWLNGKEYKCKV
SNKALPAPIEKTISKAKGQPREPQVYTLPPSRDELTKNQV
SLTCLVKGFYPSDIAVEWESNGQPENNYKTTPPVLDSDGS
FFLYSKLTVDKSRWQQGNVFSCSVMHEALHNHYTQKSLSL
SPGK
SEQ ID NO: 73:
scFV (a-CTLA4)-L-Ra (sushi)-L-Fc-L
(*)-IL15-L-Fc (1257)
METDTLLLWVLLLWVPGSTGDEIVLTQSPGTLSLSPGERA
TLSCRASQSVGSSYLAWYQQKPGQAPRLLIYGAFSRATGI
PDRFSGSGSGTDFTLTISRLEPEDFAVYYCQQYGSSPWTF
GQGTKVEIKGGGGSGGGGSGGGGSQVQLVESGGGVVQPGR
SLRLSCAASGFTFSSYTMHWVRQAPGKGLEWVTFISYDGN
NKYYADSVKGRFTISRDNSKNTLYLQMNSLRAEDTAIYYC
ARTGWLGPFDYWGQGTLVTVSSGGSGGITCPPPMSVEHAD
IWVKSYSLYSRERYICNSGFKRKAGTSSLTECVLNKATNV
AHWTTPSLKCIRDPALVHQRPAPPGGGGSEPKSSDKTHTC
PPCPAPELLGGPSVFLFPPKPKDTLMISRTPEVTCVVVDV
SHEDPEVKFNWYVDGVEVHNAKTKPREEQYNSTYRVVSVL
TVLHQDWLNGKEYKCKVSNKALPAPIEKTISKAKGQPREP
QVYTLPPSRDELTKNQVSLTCLVKGFYPSDIAVEWESNGQ
PENNYKTTPPVLDSDGSFFLYSKLTVDKSRWQQGNVFSCS
VMHEALHNHYTQKSLSLSPGKGGSSGARYRWLTAGGSGRS
ENIRTAGGSNWVNVISDLKKIEDLIQSMHIDATLYTESDV
HPSCKVTAMKCFLLELQVISLESGDASIHDTVENLIILAN
NSLSSNGNVTESGCKECEELEEKNIKEFLQSFVHIVQMFI
NTSSGGGSEPKSSDKTHTCPPCPAPELLGGPSVFLFPPKP
KDTLMISRTPEVTCVVVDVSHEDPEVKFNWYVDGVEVHNA
KTKPREEQYNSTYRVVSVLTVLHQDWLNGKEYKCKVSNKA
LPAPIEKTISKAKGQPREPQVYTLPPSRDELTKNQVSLTC
LVKGFYPSDIAVEWESNGQPENNYKTTPPVLDSDGSFFLY
SKLTVDKSRWQQGNVFSCSVMHEALHNHYTQKSLSLSPGK
SEQ ID NO: 74:
15Rb-L(*)-Ra (sushi)-L-Fc-L(*)-IL15-L-
Fc (1198B)
METDTLLLWVLLLWVPGSTGDSQFTCFYNSRANISCVWSQ
DGALQDTSCQVHAWPDRRRWNQTCELLPVSQASWACNLIL
GAPDSQKLTTVDIVTLRVLCREGVRWRVMAIQDFKPFENL
RLMAPISLQVVHVETHRCNISWEISQASHYFERHLEFEAR
TLSPGHTWEEAPLLTLKQKQEWICLETLTPDTQYEFQVRV
KPLQGEFTTWSPWSQPLAFRTKPASENIRTASGRSPAIFT
ASITCPPPMSVEHADIWVKSYSLYSRERYICNSGFKRKAG
TSSLTECVLNKATNVAHWTTPSLKCIRDPALVHQRPAPPG
GGGSEPKSSDKTHTCPPCPAPELLGGPSVFLFPPKPKDTL
MISRTPEVTCVVVDVSHEDPEVKFNWYVDGVEVHNAKTKP
REEQYNSTYRVVSVLTVLHQDWLNGKEYKCKVSNKALPAP
IEKTISKAKGQPREPQVYTLPPSRDELTKNQVSLTCLVKG
FYPSDIAVEWESNGQPENNYKTTPPVLDSDGSFFLYSKLT
VDKSRWQQGNVFSCSVMHEALHNHYTQKSLSLSPGKGGSS
GARYRWLTAGGSGRSENIRTAGGSNWVNVISDLKKIEDLI
QSMHIDATLYTESDVHPSCKVTAMKCFLLELQVISLESGD
ASIHDTVENLIILANNSLSSNGNVTESGCKECEELEEKNI
KEFLQSFVHIVQMFINTSSGGGSEPKSSDKTHTCPPCPAP
ELLGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALPAPIEKTISKAKGQPREPQVYTLP
PSRDELTKNQVSLTCLVKGFYPSDIAVEWESNGQPENNYK
TTPPVLDSDGSFFLYSKLTVDKSRWQQGNVFSCSVMHEAL
HNHYTQKSLSLSPGK
SEQ ID NO: 75:
15Rb-L (*)-Ra (sushi)-L-Fc-L(*)-IL15-L-
Fc-L-hIFNa2m (1229)
METDTLLLWVLLLWVPGSTGDSQFTCFYNSRANISCVWSQ
DGALQDTSCQVHAWPDRRRWNQTCELLPVSQASWACNLIL
GAPDSQKLTTVDIVTLRVLCREGVRWRVMAIQDFKPFENL
RLMAPISLQVVHVETHRCNISWEISQASHYFERHLEFEAR
TLSPGHTWEEAPLLTLKQKQEWICLETLTPDTQYEFQVRV
KPLQGEFTTWSPWSQPLAFRTKPASENIRTASGRSPAIFT
ASITCPPPMSVEHADIWVKSYSLYSRERYICNSGFKRKAG
TSSLTECVLNKATNVAHWTTPSLKCIRDPALVHQRPAPPG
GGGSEPKSSDKTHTCPPCPAPELLGGPSVFLFPPKPKDTL
MISRTPEVTCVVVDVSHEDPEVKFNWYVDGVEVHNAKTKP
REEQYNSTYRVVSVLTVLHQDWLNGKEYKCKVSNKALPAP
IEKTISKAKGQPREPQVYTLPPSRDELTKNQVSLTCLVKG
FYPSDIAVEWESNGQPENNYKTTPPVLDSDGSFFLYSKLT
VDKSRWQQGNVFSCSVMHEALHNHYTQKSLSLSPGKGGSS
GARYRWLTAGGSGRSENIRTAGGSNWVNVISDLKKIEDLI
QSMHIDATLYTESDVHPSCKVTAMKCFLLELQVISLESGD
ASIHDTVENLIILANNSLSSNGNVTESGCKECEELEEKNI
KEFLQSFVHIVQMFINTSSGGGSEPKSSDKTHTCPPCPAP
ELLGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALPAPIEKTISKAKGQPREPQVYTLP
PSRDELTKNQVSLTCLVKGFYPSDIAVEWESNGQPENNYK
TTPPVLDSDGSFFLYSKLTVDKSRWQQGNVFSCSVMHEAL
HNHYTQKSLSLSPGKSRPHSRGGGSGGGSTGRSCDLPQTH
SLGSRRTLMLLAQMRRISLFSCLKDRHDFGFPQEEFGNQF
QKAETIPVLHEMIQQIFNLFSTKDSSAAWDETLLDKFYTE
LYQQLNDLEACVIQGVGVTETPLMKEDSILAVRKYFRRIT
LYLKEKKYSPCAWEVVRAEIMRSFSLSTNLQESLRSKE
SEQ ID NO: 76:
Ra (sushi)-L-Fc-L(*)-IL15-L-Fc-L(*)-
15Rb (1199C)
METDTLLLWVLLLWVPGSTGDITCPPPMSVEHADIWVKSY
SLYSRERYICNSGFKRKAGTSSLTECVLNKATNVAHWTTP
SLKCIRDPALVHQRPAPPGGGGSEPKSSDKTHTCPPCPAP
ELLGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALPAPIEKTISKAKGQPREPQVYTLP
PSRDELTKNQVSLTCLVKGFYPSDIAVEWESNGQPENNYK
TTPPVLDSDGSFFLYSKLTVDKSRWQQGNVFSCSVMHEAL
HNHYTQKSLSLSPGKGGSSGARYRWLTAGGSGRSENIRTA
GGSNWVNVISDLKKIEDLIQSMHIDATLYTESDVHPSCKV
TAMKCFLLELQVISLESGDASIHDTVENLIILANNSLSSN
GNVTESGCKECEELEEKNIKEFLQSFVHIVQMFINTSSGG
GSEPKSSDKTHTCPPCPAPELLGGPSVFLFPPKPKDTLMI
SRTPEVTCVVVDVSHEDPEVKFNWYVDGVEVHNAKTKPRE
EQYNSTYRVVSVLTVLHQDWLNGKEYKCKVSNKALPAPIE
KTISKAKGQPREPQVYTLPPSRDELTKNQVSLTCLVKGFY
PSDIAVEWESNGQPENNYKTTPPVLDSDGSFFLYSKLTVD
KSRWQQGNVFSCSVMHEALHNHYTQKSLSLSPGKTGGSGR
SPAIFTAGGGSRSENIRTSGGTGGSQFTCFYNSRANISCV
WSQDGALQDTSCQVHAWPDRRRWNQTCELLPVSQASWACN
LILGAPDSQKLTTVDIVTLRVLCREGVRWRVMAIQDFKPF
ENLRLMAPISLQVVHVETHRCNISWEISQASHYFERHLEF
EARTLSPGHTWEEAPLLTLKQKQEWICLETLTPDTQYEFQ
VRVKPLQGEFTTWSPWSQPLAFRTKPAALGKDTG
SEQ ID NO: 77:
scFV (a-hPD1)-L- Ra (sushi)-L-Fc-
L(*)-IL15-L-Fc-L(*)-15Rb
(1225)
METDTLLLWVLLLWVPGSTGDEIVLTQSPATLSLSPGERA
TLSCRASKGVSTSGYSYLHWYQQKPGQAPRLLIYLASYLE
SGVPARFSGSGSGTDFTLTISSLEPEDFAVYYCQHSRDLP
LTFGGGTKVEIKRTGGGGSGGGGSGGGGSQVQLVQSGVEV
KKPGASVKVSCKASGYTFTNYYMYWVRQAPGQGLEWMGGI
NPSNGGTNFNEKFKNRVTLTTDSSTTTAYMELKSLQFDDT
AVYYCARRDYRFDMGFDYWGQGTTVTVSSGGSGGITCPPP
MSVEHADIWVKSYSLYSRERYICNSGFKRKAGTSSLTECV
LNKATNVAHWTTPSLKCIRDPALVHQRPAPPGGGGSEPKS
SDKTHTCPPCPAPELLGGPSVFLFPPKPKDTLMISRTPEV
TCVVVDVSHEDPEVKFNWYVDGVEVHNAKTKPREEQYNST
YRVVSVLTVLHQDWLNGKEYKCKVSNKALPAPIEKTISKA
KGQPREPQVYTLPPSRDELTKNQVSLTCLVKGFYPSDIAV
EWESNGQPENNYKTTPPVLDSDGSFFLYSKLTVDKSRWQQ
GNVFSCSVMHEALHNHYTQKSLSLSPGKGGSSGARYRWLT
AGGSGRSENIRTAGGSNWVNVISDLKKIEDLIQSMHIDAT
LYTESDVHPSCKVTAMKCFLLELQVISLESGDASIHDTVE
NLIILANNSLSSNGNVTESGCKECEELEEKNIKEFLQSFV
HIVQMFINTSSGGGSEPKSSDKTHTCPPCPAPELLGGPSV
FLFPPKPKDTLMISRTPEVTCVVVDVSHEDPEVKFNWYVD
GVEVHNAKTKPREEQYNSTYRVVSVLTVLHQDWLNGKEYK
CKVSNKALPAPIEKTISKAKGQPREPQVYTLPPSRDELTK
NQVSLTCLVKGFYPSDIAVEWESNGQPENNYKTTPPVLDS
DGSFFLYSKLTVDKSRWQQGNVFSCSVMHEALHNHYTQKS
LSLSPGKTGGSGRSPAIFTAGGGSRSENIRTSGGTGGSQF
TCFYNSRANISCVWSQDGALQDTSCQVHAWPDRRRWNQTC
ELLPVSQASWACNLILGAPDSQKLTTVDIVTLRVLCREGV
RWRVMAIQDFKPFENLRLMAPISLQVVHVETHRCNISWEI
SQASHYFERHLEFEARTLSPGHTWEEAPLLTLKQKQEWIC
LETLTPDTQYEFQVRVKPLQGEFTTWSPWSQPLAFRTKPA
ALGKDTG
SEQ ID NO: 78:
hIFNam2-L- Ra (sushi)-L-Fc-L(*)-IL15-
L-Fc-L(*)-15Rb (1226)
METDTLLLWVLLLWVPGSTGDCDLPQTHSLGSRRTLMLLA
QMRRISLFSCLKDRHDFGFPQEEFGNQFQKAETIPVLHEM
IQQIFNLFSTKDSSAAWDETLLDKFYTELYQQLNDLEACV
IQGVGVTETPLMKEDSILAVRKYFRRITLYLKEKKYSPCA
WEVVRAEIMRSFSLSTNLQESLRSKESGGGSITCPPPMSV
EHADIWVKSYSLYSRERYICNSGFKRKAGTSSLTECVLNK
ATNVAHWTTPSLKCIRDPALVHQRPAPPGGGGSEPKSSDK
THTCPPCPAPELLGGPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSRDELTKNQVSLTCLVKGFYPSDIAVEWE
SNGQPENNYKTTPPVLDSDGSFFLYSKLTVDKSRWQQGNV
FSCSVMHEALHNHYTQKSLSLSPGKGGSSGARYRWLTAGG
SGRSENIRTAGGSNWVNVISDLKKIEDLIQSMHIDATLYT
ESDVHPSCKVTAMKCFLLELQVISLESGDASIHDTVENLI
ILANNSLSSNGNVTESGCKECEELEEKNIKEFLQSFVHIV
QMFINTSSGGGSEPKSSDKTHTCPPCPAPELLGGPSVFLF
PPKPKDTLMISRTPEVTCVVVDVSHEDPEVKFNWYVDGVE
VHNAKTKPREEQYNSTYRVVSVLTVLHQDWLNGKEYKCKV
SNKALPAPIEKTISKAKGQPREPQVYTLPPSRDELTKNQV
SLTCLVKGFYPSDIAVEWESNGQPENNYKTTPPVLDSDGS
FFLYSKLTVDKSRWQQGNVFSCSVMHEALHNHYTQKSLSL
SPGKTGGSGRSPAIFTAGGGSRSENIRTSGGTGGSQFTCF
YNSRANISCVWSQDGALQDTSCQVHAWPDRRRWNQTCELL
PVSQASWACNLILGAPDSQKLTTVDIVTLRVLCREGVRWR
VMAIQDFKPFENLRLMAPISLQVVHVETHRCNISWEISQA
SHYFERHLEFEARTLSPGHTWEEAPLLTLKQKQEWICLET
LTPDTQYEFQVRVKPLQGEFTTWSPWSQPLAFRTKPAALG
KDTG
SEQ ID NO: 79:
scFV (a-hCTLA4)-L- Ra (sushi)-L-Fc-
L(*)-IL15-L-Fc-L(*)-15Rb
(1232)
METDTLLLWVLLLWVPGSTGDEIVLTQSPGTLSLSPGERA
TLSCRASQSVGSSYLAWYQQKPGQAPRLLIYGAFSRATGI
PDRFSGSGSGTDFTLTISRLEPEDFAVYYCQQYGSSPWTF
GQGTKVEIKGGGGSGGGGSGGGGSQVQLVESGGGVVQPGR
SLRLSCAASGFTFSSYTMHWVRQAPGKGLEWVTFISYDGN
NKYYADSVKGRFTISRDNSKNTLYLQMNSLRAEDTAIYYC
ARTGWLGPFDYWGQGTLVTVSSGGSGGITCPPPMSVEHAD
IWVKSYSLYSRERYICNSGFKRKAGTSSLTECVLNKATNV
AHWTTPSLKCIRDPALVHQRPAPPGGGGSEPKSSDKTHTC
PPCPAPELLGGPSVFLFPPKPKDTLMISRTPEVTCVVVDV
SHEDPEVKFNWYVDGVEVHNAKTKPREEQYNSTYRVVSVL
TVLHQDWLNGKEYKCKVSNKALPAPIEKTISKAKGQPREP
QVYTLPPSRDELTKNQVSLTCLVKGFYPSDIAVEWESNGQ
PENNYKTTPPVLDSDGSFFLYSKLTVDKSRWQQGNVFSCS
VMHEALHNHYTQKSLSLSPGKGGSSGARYRWLTAGGSGRS
ENIRTAGGSNWVNVISDLKKIEDLIQSMHIDATLYTESDV
HPSCKVTAMKCFLLELQVISLESGDASIHDTVENLIILAN
NSLSSNGNVTESGCKECEELEEKNIKEFLQSFVHIVQMFI
NTSSGGGSEPKSSDKTHTCPPCPAPELLGGPSVFLFPPKP
KDTLMISRTPEVTCVVVDVSHEDPEVKFNWYVDGVEVHNA
KTKPREEQYNSTYRVVSVLTVLHQDWLNGKEYKCKVSNKA
LPAPIEKTISKAKGQPREPQVYTLPPSRDELTKNQVSLTC
LVKGFYPSDIAVEWESNGQPENNYKTTPPVLDSDGSFFLY
SKLTVDKSRWQQGNVFSCSVMHEALHNHYTQKSLSLSPGK
TGGSGRSPAIFTAGGGSRSENIRTSGGTGGSQFTCFYNSR
ANISCVWSQDGALQDTSCQVHAWPDRRRWNQTCELLPVSQ
ASWACNLILGAPDSQKLTTVDIVTLRVLCREGVRWRVMAI
QDFKPFENLRLMAPISLQVVHVETHRCNISWEISQASHYF
ERHLEFEARTLSPGHTWEEAPLLTLKQKQEWICLETLTPD
TQYEFQVRVKPLQGEFTTWSPWSQPLAFRTKPAALGKDTG
SEQ ID NO: 80:
hIL12P40-L-Fc-L(*)-hIL12P35-L-Fc (IT25)
METDTLLLWVLLLWVPGSTGDIWELKKDVYVVELDWYPDA
PGEMVVLTCDTPEEDGITWTLDQSSEVLGSGKTLTIQVKE
FGDAGQYTCHKGGEVLSHSLLLLHKKEDGIWSTDILKDQK
EPKNKTFLRCEAKNYSGRFTCWWLTTISTDLTFSVKSSRG
SSDPQGVTCGAATLSAERVRGDNKEYEYSVECQEDSACPA
AEESLPIEVMVDAVHKLKYENYTSSFFIRDIIKPDPPKNL
QLKPLKNSRQVEVSWEYPDTWSTPHSYFSLTFCVQVQGKS
KREKKDRVFTDKTSATVICRKNASISVRAQDRYYSSSWSE
WASVPCSEPKSSDKTHTCPPCPAPEAAGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
GAPIEKTISKAKGQPREPQVYTLPPSRDELTKNQVSLTCL
VKGFYPSDIAVEWESNGQPENNYKTTPPVLDSDGSFFLYS
KLTVDKSRWQQGNVFSCSVMHEALHNHYTQKSLSLSPGKL
EGGGGSGRSENIRTATGGGGSGGGSGTRNLPVATPDPGMF
PCLHHSQNLLRAVSNMLQKARQTLEFYPCTSEEIDHEDIT
KDKTSTVEACLPLELTKNESCLNSRETSFITNGSCLASRK
TSFMMALCLSSIYEDLKMYQVEFKTMNAKLLMDPKRQIFL
DQNMLAVIDELMQALNFNSETVPQKSSLEEPDFYKTKIKL
CILLHAFRIRAVTIDRVMSYLNASEPKSSDKTHTCPPCPA
PEAAGGPSVFLFPPKPKDTLMISRTPEVTCVVVDVSHEDP
EVKFNWYVDGVEVHNAKTKPREEQYNSTYRVVSVLTVLHQ
DWLNGKEYKCKVSNKALGAPIEKTISKAKGQPREPQVYTL
PPSRDELTKNQVSLTCLVKGFYPSDIAVEWESNGQPENNY
KTTPPVLDSDGSFFLYSKLTVDKSRWQQGNVFSCSVMHEA
LHNHYTQKSLSLSPGK
SEQ ID NO: 81:
12Rbl-L(*)-hIL12P40-L-Fc-L(*)-
hIL12P35-L-Fc (IT30)
METDTLLLWVLLLWVPGSTGDCRTECCFQDPPYPDADSGS
ASGPRDLRCYRISSDRYECSWQYEGPTAGVSHFLRCCLSS
GRCCYFAAGSATRLQFSDQAGVSVLYTVTLWVESWARNQT
EKSPEVTLQLYNSVKYEPPLGDIKVSKLAGQLRMEWETPD
NQVGAEVQFRHRTPSSPWKLGDCGPQDDDTESCLCPLEMN
VAQEFQLRRRQLGSQGSSWSKWSSPVCVPPENLEGGGGSG
RSENIRTATGGGGSGGGSGTIWELKKDVYVVELDWYPDAP
GEMVVLTCDTPEEDGITWTLDQSSEVLGSGKTLTIQVKEF
GDAGQYTCHKGGEVLSHSLLLLHKKEDGIWSTDILKDQKE
PKNKTFLRCEAKNYSGRFTCWWLTTISTDLTFSVKSSRGS
SDPQGVTCGAATLSAERVRGDNKEYEYSVECQEDSACPAA
EESLPIEVMVDAVHKLKYENYTSSFFIRDIIKPDPPKNLQ
LKPLKNSRQVEVSWEYPDTWSTPHSYFSLTFCVQVQGKSK
REKKDRVFTDKTSATVICRKNASISVRAQDRYYSSSWSEW
ASVPCSEPKSSDKTHTCPPCPAPEAAGGPSVFLFPPKPKD
TLMISRTPEVTCVVVDVSHEDPEVKFNWYVDGVEVHNAKT
KPREEQYNSTYRVVSVLTVLHQDWLNGKEYKCKVSNKALG
APIEKTISKAKGQPREPQVYTLPPSRDELTKNQVSLTCLV
KGFYPSDIAVEWESNGQPENNYKTTPPVLDSDGSFFLYSK
LTVDKSRWQQGNVFSCSVMHEALHNHYTQKSLSLSPGKLE
GGGGSGRSENIRTATGGGGSGGGSGTRNLPVATPDPGMFP
CLHHSQNLLRAVSNMLQKARQTLEFYPCTSEEIDHEDITK
DKTSTVEACLPLELTKNESCLNSRETSFITNGSCLASRKT
SFMMALCLSSIYEDLKMYQVEFKTMNAKLLMDPKRQIFLD
QNMLAVIDELMQALNFNSETVPQKSSLEEPDFYKTKIKLC
ILLHAFRIRAVTIDRVMSYLNASEPKSSDKTHTCPPCPAP
EAAGGPSVFLFPPKPKDTLMISRTPEVTCVVVDVSHEDPE
VKFNWYVDGVEVHNAKTKPREEQYNSTYRVVSVLTVLHQD
WLNGKEYKCKVSNKALGAPIEKTISKAKGQPREPQVYTLP
PSRDELTKNQVSLTCLVKGFYPSDIAVEWESNGQPENNYK
TTPPVLDSDGSFFLYSKLTVDKSRWQQGNVFSCSVMHEAL
HNHYTQKSLSLSPGK
SEQ ID NO: 82:
hIL12P40-L-Fc- L2(*)-hIL122P35-L-
Fc-L (*)-12Rb2 (IT32)
METDTLLLWVLLLWVPGSTGDIWELKKDVYVVELDWYPDA
PGEMVVLTCDTPEEDGITWTLDQSSEVLGSGKTLTIQVKE
FGDAGQYTCHKGGEVLSHSLLLLHKKEDGIWSTDILKDQK
EPKNKTFLRCEAKNYSGRFTCWWLTTISTDLTFSVKSSRG
SSDPQGVTCGAATLSAERVRGDNKEYEYSVECQEDSACPA
AEESLPIEVMVDAVHKLKYENYTSSFFIRDIIKPDPPKNL
QLKPLKNSRQVEVSWEYPDTWSTPHSYFSLTFCVQVQGKS
KREKKDRVFTDKTSATVICRKNASISVRAQDRYYSSSWSE
WASVPCSEPKSSDKTHTCPPCPAPEAAGGPSVFLFPPKPK
DTLMISRTPEVTCVVVDVSHEDPEVKFNWYVDGVEVHNAK
TKPREEQYNSTYRVVSVLTVLHQDWLNGKEYKCKVSNKAL
GAPIEKTISKAKGQPREPQVYTLPPSRDELTKNQVSLTCL
VKGFYPSDIAVEWESNGQPENNYKTTPPVLDSDGSFFLYS
KLTVDKSRWQQGNVFSCSVMHEALHNHYTQKSLSLSPGKL
EGGGGSGRSENIRTATGGGGSGGGSGTRNLPVATPDPGMF
PCLHHSQNLLRAVSNMLQKARQTLEFYPCTSEEIDHEDIT
KDKTSTVEACLPLELTKNESCLNSRETSFITNGSCLASRK
TSFMMALCLSSIYEDLKMYQVEFKTMNAKLLMDPKRQIFL
DQNMLAVIDELMQALNFNSETVPQKSSLEEPDFYKTKIKL
CILLHAFRIRAVTIDRVMSYLNASGGGSEPKSSDKTHTCP
PCPAPEAAGGPSVFLFPPKPKDTLMISRTPEVTCVVVDVS
HEDPEVKFNWYVDGVEVHNAKTKPREEQYNSTYRVVSVLT
VLHQDWLNGKEYKCKVSNKALGAPIEKTISKAKGQPREPQ
VYTLPPSRDELTKNQVSLTCLVKGFYPSDIAVEWESNGQP
ENNYKTTPPVLDSDGSFFLYSKLTVDKSRWQQGNVFSCSV
MHEALHNHYTQKSLSLSPGKGGGSSGARYRWLTAGGSGRA
MHMYTAGGRSGGIDACKRGDVTVKPSHVILLGSTVNITCS
LKPRQGCFHYSRRNKLILYKFDRRINFHHGHSLNSQVTGL
PLGTTLFVCKLACINSDEIQICGAEIFVGVAPEQPQNLSC
IQKGEQGTVACTWERGRDTHLYTEYTLQLSGPKNLTWQKQ
CKDIYCDYLDFGINLTPESPESNFTAKVTAVNSLGSSSSL
PSTFTFLDIVRPLPPWDIRIKFQKASVSRCTLYWRDEGLV
LLNRLRYRPSNSRLWNMVNVTKAKGRHDLLDLKPFTEYEF
QISSKLHLYKGSWSDWSESLRAQTPEE
SEQ ID NO: 83:
12Rb1-L (*)-hIL12P40-L-Fc-L (*)-
hIL12P35-L-Fc-L (*)-12Rb2
(IT33)
METDTLLLWVLLLWVPGSTGDCRTECCFQDPPYPDADSGS
ASGPRDLRCYRISSDRYECSWQYEGPTAGVSHFLRCCLSS
GRCCYFAAGSATRLQFSDQAGVSVLYTVTLWVESWARNQT
EKSPEVTLQLYNSVKYEPPLGDIKVSKLAGQLRMEWETPD
NQVGAEVQFRHRTPSSPWKLGDCGPQDDDTESCLCPLEMN
VAQEFQLRRRQLGSQGSSWSKWSSPVCVPPENLEGGGGSG
RSENIRTATGGGGSGGGSGTIWELKKDVYVVELDWYPDAP
GEMVVLTCDTPEEDGITWTLDQSSEVLGSGKTLTIQVKEF
GDAGQYTCHKGGEVLSHSLLLLHKKEDGIWSTDILKDQKE
PKNKTFLRCEAKNYSGRFTCWWLTTISTDLTFSVKSSRGS
SDPQGVTCGAATLSAERVRGDNKEYEYSVECQEDSACPAA
EESLPIEVMVDAVHKLKYENYTSSFFIRDIIKPDPPKNLQ
LKPLKNSRQVEVSWEYPDTWSTPHSYFSLTFCVQVQGKSK
REKKDRVFTDKTSATVICRKNASISVRAQDRYYSSSWSEW
ASVPCSEPKSSDKTHTCPPCPAPEAAGGPSVFLFPPKPKD
TLMISRTPEVTCVVVDVSHEDPEVKFNWYVDGVEVHNAKT
KPREEQYNSTYRVVSVLTVLHQDWLNGKEYKCKVSNKALG
APIEKTISKAKGQPREPQVYTLPPSRDELTKNQVSLTCLV
KGFYPSDIAVEWESNGQPENNYKTTPPVLDSDGSFFLYSK
LTVDKSRWQQGNVFSCSVMHEALHNHYTQKSLSLSPGKLE
GGGGSGRSENIRTATGGGGSGGGSGTRNLPVATPDPGMFP
CLHHSQNLLRAVSNMLQKARQTLEFYPCTSEEIDHEDITK
DKTSTVEACLPLELTKNESCLNSRETSFITNGSCLASRKT
SFMMALCLSSIYEDLKMYQVEFKTMNAKLLMDPKRQIFLD
QNMLAVIDELMQALNFNSETVPQKSSLEEPDFYKTKIKLC
ILLHAFRIRAVTIDRVMSYLNASGGGSEPKSSDKTHTCPP
CPAPEAAGGPSVFLFPPKPKDTLMISRTPEVTCVVVDVSH
EDPEVKFNWYVDGVEVHNAKTKPREEQYNSTYRVVSVLTV
LHQDWLNGKEYKCKVSNKALGAPIEKTISKAKGQPREPQV
YTLPPSRDELTKNQVSLTCLVKGFYPSDIAVEWESNGQPE
NNYKTTPPVLDSDGSFFLYSKLTVDKSRWQQGNVFSCSVM
HEALHNHYTQKSLSLSPGKGGGSSGARYRWLTAGGSGRAM
HMYTAGGRSGGIDACKRGDVTVKPSHVILLGSTVNITCSL
KPRQGCFHYSRRNKLILYKFDRRINFHHGHSLNSQVTGLP
LGTTLFVCKLACINSDEIQICGAEIFVGVAPEQPQNLSCI
QKGEQGTVACTWERGRDTHLYTEYTLQLSGPKNLTWQKQC
KDIYCDYLDFGINLTPESPESNFTAKVTAVNSLGSSSSLP
STFTFLDIVRPLPPWDIRIKFQKASVSRCTLYWRDEGLVL
LNRLRYRPSNSRLWNMVNVTKAKGRHDLLDLKPFTEYEFQ
ISSKLHLYKGSWSDWSESLRAQTPEE
SEQ ID NO: 84:
VL-L-VH (a-hPD1)-L-hIL12P40-L-Fc-L
(*)-hIL12P35-L-Fc-L(*)-12Rb2 (IT43)
METDTLLLWVLLLWVPGSTGDEIVLTQSPATLSLSPGERA
TLSCRASKGVSTSGYSYLHWYQQKPGQAPRLLIYLASYLE
SGVPARFSGSGSGTDFTLTISSLEPEDFAVYYCQHSRDLP
LTFGGGTKVEIKRTGGGGSGGGGSGGGGSQVQLVQSGVEV
KKPGASVKVSCKASGYTFTNYYMYWVRQAPGQGLEWMGGI
NPSNGGTNFNEKFKNRVTLTTDSSTTTAYMELKSLQFDDT
AVYYCARRDYRFDMGFDYWGQGTTVTVSSGGSRFESGGSI
WELKKDVYVVELDWYPDAPGEMVVLTCDTPEEDGITWTLD
QSSEVLGSGKTLTIQVKEFGDAGQYTCHKGGEVLSHSLLL
LHKKEDGIWSTDILKDQKEPKNKTFLRCEAKNYSGRFTCW
WLTTISTDLTFSVKSSRGSSDPQGVTCGAATLSAERVRGD
NKEYEYSVECQEDSACPAAEESLPIEVMVDAVHKLKYENY
TSSFFIRDIIKPDPPKNLQLKPLKNSRQVEVSWEYPDTWS
TPHSYFSLTFCVQVQGKSKREKKDRVFTDKTSATVICRKN
ASISVRAQDRYYSSSWSEWASVPCSEPKSSDKTHTCPPCP
APEAAGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALGAPIEKTISKAKGQPREPQVYT
LPPSRDELTKNQVSLTCLVKGFYPSDIAVEWESNGQPENN
YKTTPPVLDSDGSFFLYSKLTVDKSRWQQGNVFSCSVMHE
ALHNHYTQKSLSLSPGKLEGGGGSGRSENIRTATGGGGSG
GGSGTRNLPVATPDPGMFPCLHHSQNLLRAVSNMLQKARQ
TLEFYPCTSEEIDHEDITKDKTSTVEACLPLELTKNESCL
NSRETSFITNGSCLASRKTSFMMALCLSSIYEDLKMYQVE
FKTMNAKLLMDPKRQIFLDQNMLAVIDELMQALNFNSETV
PQKSSLEEPDFYKTKIKLCILLHAFRIRAVTIDRVMSYLN
ASGGGSEPKSSDKTHTCPPCPAPEAAGGPSVFLFPPKPKD
TLMISRTPEVTCVVVDVSHEDPEVKFNWYVDGVEVHNAKT
KPREEQYNSTYRVVSVLTVLHQDWLNGKEYKCKVSNKALG
APIEKTISKAKGQPREPQVYTLPPSRDELTKNQVSLTCLV
KGFYPSDIAVEWESNGQPENNYKTTPPVLDSDGSFFLYSK
LTVDKSRWQQGNVFSCSVMHEALHNHYTQKSLSLSPGKGG
GSSGARYRWLTAGGSGRAMHMYTAGGRSGGIDACKRGDVT
VKPSHVILLGSTVNITCSLKPRQGCFHYSRRNKLILYKFD
RRINFHHGHSLNSQVTGLPLGTTLFVCKLACINSDEIQIC
GAEIFVGVAPEQPQNLSCIQKGEQGTVACTWERGRDTHLY
TEYTLQLSGPKNLTWQKQCKDIYCDYLDFGINLTPESPES
NFTAKVTAVNSLGSSSSLPSTFTFLDIVRPLPPWDIRIKF
QKASVSRCTLYWRDEGLVLLNRLRYRPSNSRLWNMVNVTK
AKGRHDLLDLKPFTEYEFQISSKLHLYKGSWSDWSESLRA
QTPEE
SEQ ID NO: 85:
VL-L-VH (a-hPD1)-L-IL-12hP40-L-Fc-L(*)-
hIL12P35-L-Fc (IT46)
METDTLLLWVLLLWVPGSTGDEIVLTQSPATLSLSPGERA
TLSCRASKGVSTSGYSYLHWYQQKPGQAPRLLIYLASYLE
SGVPARFSGSGSGTDFTLTISSLEPEDFAVYYCQHSRDLP
LTFGGGTKVEIKRTGGGGSGGGGSGGGGSQVQLVQSGVEV
KKPGASVKVSCKASGYTFTNYYMYWVRQAPGQGLEWMGGI
NPSNGGTNFNEKFKNRVTLTTDSSTTTAYMELKSLQFDDT
AVYYCARRDYRFDMGFDYWGQGTTVTVSSGGSRFESGGSI
WELKKDVYVVELDWYPDAPGEMVVLTCDTPEEDGITWTLD
QSSEVLGSGKTLTIQVKEFGDAGQYTCHKGGEVLSHSLLL
LHKKEDGIWSTDILKDQKEPKNKTFLRCEAKNYSGRFTCW
WLTTISTDLTFSVKSSRGSSDPQGVTCGAATLSAERVRGD
NKEYEYSVECQEDSACPAAEESLPIEVMVDAVHKLKYENY
TSSFFIRDIIKPDPPKNLQLKPLKNSRQVEVSWEYPDTWS
TPHSYFSLTFCVQVQGKSKREKKDRVFTDKTSATVICRKN
ASISVRAQDRYYSSSWSEWASVPCSEPKSSDKTHTCPPCP
APEAAGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALGAPIEKTISKAKGQPREPQVYT
LPPSRDELTKNQVSLTCLVKGFYPSDIAVEWESNGQPENN
YKTTPPVLDSDGSFFLYSKLTVDKSRWQQGNVFSCSVMHE
ALHNHYTQKSLSLSPGKLEGGGGSGRSENIRTATGGGGSG
GGSGTRNLPVATPDPGMFPCLHHSQNLLRAVSNMLQKARQ
TLEFYPCTSEEIDHEDITKDKTSTVEACLPLELTKNESCL
NSRETSFITNGSCLASRKTSFMMALCLSSIYEDLKMYQVE
FKTMNAKLLMDPKRQIFLDQNMLAVIDELMQALNFNSETV
PQKSSLEEPDFYKTKIKLCILLHAFRIRAVTIDRVMSYLN
ASGGGSEPKSSDKTHTCPPCPAPEAAGGPSVFLFPPKPKD
TLMISRTPEVTCVVVDVSHEDPEVKFNWYVDGVEVHNAKT
KPREEQYNSTYRVVSVLTVLHQDWLNGKEYKCKVSNKALG
APIEKTISKAKGQPREPQVYTLPPSRDELTKNQVSLTCLV
KGFYPSDIAVEWESNGQPENNYKTTPPVLDSDGSFFLYSK
LTVDKSRWQQGNVFSCSVMHEALHNHYTQKSLSLSPGK
SEQ ID NO: 86:
VL-L-VH (a-hPD1)-L-Fc-L-hIL-12P40-L(*)-
Fc-L-hIL12P35 (IT49)
METDTLLLWVLLLWVPGSTGDEIVLTQSPATLSLSPGERA
TLSCRASKGVSTSGYSYLHWYQQKPGQAPRLLIYLASYLE
SGVPARFSGSGSGTDFTLTISSLEPEDFAVYYCQHSRDLP
LTFGGGTKVEIKRTGGGGSGGGGSGGGGSQVQLVQSGVEV
KKPGASVKVSCKASGYTFTNYYMYWVRQAPGQGLEWMGGI
NPSNGGTNFNEKFKNRVTLTTDSSTTTAYMELKSLQFDDT
AVYYCARRDYRFDMGFDYWGQGTTVTVSSEPKSSDKTHTC
PPCPAPEAAGGPSVFLFPPKPKDTLMISRTPEVTCVVVDV
SHEDPEVKFNWYVDGVEVHNAKTKPREEQYNSTYRVVSVL
TVLHQDWLNGKEYKCKVSNKALGAPIEKTISKAKGQPREP
QVYTLPPSRDELTKNQVSLTCLVKGFYPSDIAVEWESNGQ
PENNYKTTPPVLDSDGSFFLYSKLTVDKSRWQQGNVFSCS
VMHEALHNHYTQKSLSLSPGKGGSRFESGGSIWELKKDVY
VVELDWYPDAPGEMVVLTCDTPEEDGITWTLDQSSEVLGS
GKTLTIQVKEFGDAGQYTCHKGGEVLSHSLLLLHKKEDGI
WSTDILKDQKEPKNKTFLRCEAKNYSGRFTCWWLTTISTD
LTFSVKSSRGSSDPQGVTCGAATLSAERVRGDNKEYEYSV
ECQEDSACPAAEESLPIEVMVDAVHKLKYENYTSSFFIRD
IIKPDPPKNLQLKPLKNSRQVEVSWEYPDTWSTPHSYFSL
TFCVQVQGKSKREKKDRVFTDKTSATVICRKNASISVRAQ
DRYYSSSWSEWASVPCSGGSSGARYRWLTAGGSGRSENIR
TGGGSEPKSSDKTHTCPPCPAPEAAGGPSVFLFPPKPKDT
LMISRTPEVTCVVVDVSHEDPEVKFNWYVDGVEVHNAKTK
PREEQYNSTYRVVSVLTVLHQDWLNGKEYKCKVSNKALGA
PIEKTISKAKGQPREPQVYTLPPSRDELTKNQVSLTCLVK
GFYPSDIAVEWESNGQPENNYKTTPPVLDSDGSFFLYSKL
TVDKSRWQQGNVFSCSVMHEALHNHYTQKSLSLSPGKSRP
HSRGGGSGGGSTGRSRNLPVATPDPGMFPCLHHSQNLLRA
VSNMLQKARQTLEFYPCTSEEIDHEDITKDKTSTVEACLP
LELTKNESCLNSRETSFITNGSCLASRKTSFMMALCLSSI
YEDLKMYQVEFKTMNAKLLMDPKRQIFLDQNMLAVIDELM
QALNFNSETVPQKSSLEEPDFYKTKIKLCILLHAFRIRAV
TIDRVMSYLNAS
SEQ ID NO: 87:
VL-L-VH (a-hPD1)-Fc-L-hIL-12P40-L(*)-
Fc-L-hP35-L(*)-12Rb2
(IT50)
METDTLLLWVLLLWVPGSTGDEIVLTQSPATLSLSPGERA
TLSCRASKGVSTSGYSYLHWYQQKPGQAPRLLIYLASYLE
SGVPARFSGSGSGTDFTLTISSLEPEDFAVYYCQHSRDLP
LTFGGGTKVEIKRTGGGGSGGGGSGGGGSQVQLVQSGVEV
KKPGASVKVSCKASGYTFTNYYMYWVRQAPGQGLEWMGGI
NPSNGGTNFNEKFKNRVTLTTDSSTTTAYMELKSLQFDDT
AVYYCARRDYRFDMGFDYWGQGTTVTVSSEPKSSDKTHTC
PPCPAPEAAGGPSVFLFPPKPKDTLMISRTPEVTCVVVDV
SHEDPEVKFNWYVDGVEVHNAKTKPREEQYNSTYRVVSVL
TVLHQDWLNGKEYKCKVSNKALGAPIEKTISKAKGQPREP
QVYTLPPSRDELTKNQVSLTCLVKGFYPSDIAVEWESNGQ
PENNYKTTPPVLDSDGSFFLYSKLTVDKSRWQQGNVFSCS
VMHEALHNHYTQKSLSLSPGKGGSRFESGGSIWELKKDVY
VVELDWYPDAPGEMVVLTCDTPEEDGITWTLDQSSEVLGS
GKTLTIQVKEFGDAGQYTCHKGGEVLSHSLLLLHKKEDGI
WSTDILKDQKEPKNKTFLRCEAKNYSGRFTCWWLTTISTD
LTFSVKSSRGSSDPQGVTCGAATLSAERVRGDNKEYEYSV
ECQEDSACPAAEESLPIEVMVDAVHKLKYENYTSSFFIRD
IIKPDPPKNLQLKPLKNSRQVEVSWEYPDTWSTPHSYFSL
TFCVQVQGKSKREKKDRVFTDKTSATVICRKNASISVRAQ
DRYYSSSWSEWASVPCSGGSSGARYRWLTAGGSGRSENIR
TGGGSEPKSSDKTHTCPPCPAPEAAGGPSVFLFPPKPKDT
LMISRTPEVTCVVVDVSHEDPEVKFNWYVDGVEVHNAKTK
PREEQYNSTYRVVSVLTVLHQDWLNGKEYKCKVSNKALGA
PIEKTISKAKGQPREPQVYTLPPSRDELTKNQVSLTCLVK
GFYPSDIAVEWESNGQPENNYKTTPPVLDSDGSFFLYSKL
TVDKSRWQQGNVFSCSVMHEALHNHYTQKSLSLSPGKSRP
HSRGGGSGGGSTGRSRNLPVATPDPGMFPCLHHSQNLLRA
VSNMLQKARQTLEFYPCTSEEIDHEDITKDKTSTVEACLP
LELTKNESCLNSRETSFITNGSCLASRKTSFMMALCLSSI
YEDLKMYQVEFKTMNAKLLMDPKRQIFLDQNMLAVIDELM
QALNFNSETVPQKSSLEEPDFYKTKIKLCILLHAFRIRAV
TIDRVMSYLNASGGGSSGARYRWLTAGGSGRAMHMYTAGG
RSGGIDACKRGDVTVKPSHVILLGSTVNITCSLKPRQGCF
HYSRRNKLILYKFDRRINFHHGHSLNSQVTGLPLGTTLFV
CKLACINSDEIQICGAEIFVGVAPEQPQNLSCIQKGEQGT
VACTWERGRDTHLYTEYTLQLSGPKNLTWQKQCKDIYCDY
LDFGINLTPESPESNFTAKVTAVNSLGSSSSLPSTFTFLD
IVRPLPPWDIRIKFQKASVSRCTLYWRDEGLVLLNRLRYR
PSNSRLWNMVNVTKAKGRHDLLDLKPFTEYEFQISSKLHL
YKGSWSDWSESLRAQTPEE
SEQ ID NO: 88:
human IL-2
APTSSSTKKTQLQLEHLLLDLQMILNGINNYKNPKLTRML
TFKFYMPKKATELKHLQCLEEELKPLEEVLNLAQSKNFHL
RPRDLISNINVIVLELKGSETTFMCEYADETATIVEFLNR
WITFCQSIISTLT
SEQ ID NO: 89:
human IL-7
DCDIEGKDGKQYESVLMVSIDQLLDSMKEIGSNCLNNEFN
FFKRHICDANKEGMFLFRAARKLRQFLKMNSTGDFDLHLL
KVSEGTTILLNCTGQVKGRKPAALGEAQPTKSLEENKSLK
EQKKLNDLCFLKRLLQEIKTCWNKILMGTKEH
SEQ ID NO: 90:
human IL-18
YFGKLESKLSVIRNLNDQVLFIDQGNRPLFEDMTDSDCRD
NAPRTIFIISMYKDSQPRGMAVTISVKCEKISTLSCENKI
ISFKEMNPPDNIKDTKSDIIFFQRSVPGHDNKMQFESSSY
EGYFLACEKERDLFKLILKKEDELGDRSIMFTVQNED
SEQ ID NO: 91:
human IL-21
HKSSSQGQDRHMIRMRQLIDIVDQLKNYVNDLVPEFLPAP
EDVETNCEWSAFSCFQKAQLKSANTGNNERIINVSIKKLK
RKPPSTNAGRRQKHRLTCPSCDSYEKKPPKEFLERFKSLL
QKMIHQHLSSRTHGSEDS
SEQ ID NO: 92:
anti-hu-CLDN VH-CH version 1
QVQLVQSGAEVKKPGSSVKVSCKASGYAFSNYLIEWVKQA
PGQGLEWIGLINPGSGGTNYNEKFKGKATITADKSTSTAY
MELSSLRSEDTAVYYCARVYYGNSFAYWGQGTLVTVSSAS
TKGPSVFPLAPSSKSTSGGTAALGCLVKDYFPEPVTVSWN
SGALTSGVHTFPAVLQSSGLYSLSSVVTVPSSSLGTQT
SEQ ID NO: 93:
anti-hu-CLDN VL-CL version 1
DIVMTQSPDSLAVSLGERATISCKSSQSLLNSGNQKNYLT
WYQQKPGQPPKLLIYWASTRESGVPDRFSGSGSGTDFTLT
ISSLQAEDVAVYYCQNDYFYPFTFGQGTKLEIKRTVAAPS
VFIFPPSDEQLKSGTASVVCLLNNFYPREAKVQWKVDNAL
QSGNSQESVTEQDSKDSTYSLSSTLTLSKADYEKHKVYAC
EVTHQGLSSPVTKSFNRGEC
SEQ ID NO: 94:
anti-hu-CLDN VH-CH version 2QV
QLQQPGAELVRPGASVKLSCKASGYTFTSYWINWVKQRPG
QGLEWIGNIYPSDSYTNYNQKFKDKATLTVDKSSSTAYMQ
LSSPTSEDSAVYYCARVYYGNSFAYWGQGTTLTVSSASTK
GPSVFPLAPSSKSTSGGTAALGCLVKDYFPEPVTVSWNSG
ALTSGVHTFPAVLQSSGLYSLSSVVTVPSSSLGTQTYICN
VNHKPSNTKVDKRVEPKSC
SEQ ID NO: 95:
anti-hu-CLDN VL-CL version 2
METDTLLLWVLLLWVPGSTGDIVMTQSPSSLTVTAGEKVT
MSCKSSQSLLNSGNQKNYLTWYQQKPGQPPKLLIYWASTR
ESGVPDRFTGSGSGTDFTLTISSVQAEDLAVYYCQNDYFY
PFTFGSGTKLEIKRTVAAPSVFIFPPSDEQLKSGTASVVC
LLNNFYPREAKVQWKVDNALQSGNSQESVTEQDSKDSTYS
LSSTLTLSKADYEKHKVYACEVTHQGLSSPVTKSFNRGEC
SEQ ID NO: 96:
human IL-12 P35 subunit mutein 1
(no signal peptide)
RNLPVATPDPGMFPCLHHSQNLLRAVSNMLQKARQTLEFY
PCTSEEIDHEDITKDKTSTVEACLPLELTKNESCLNSRET
SFITNGSCLASRKTSFMMALCLSSIYEDLKMYQVEFKTMN
AKLLMDPKRQIFLDQNMLAVIDELMQALNFNSETVPQKSS
LEEPDFAKTAIKLCILLHAFRIRAVTIDRVMSYLNAS
SEQ ID NO: 97:
human IL-12 P35 subunit mutein 2
(no signal peptide)
RNLPVATPDPGMFPCLHHSQNLLRAVSNMLQKARQTLEFY
PCTSEEIDHEDITKDKTSTVEACLPLELTKNESCLNSRET
SFITNGSCLASRKTSFMMALCLSSIYEDLKMYQVEFKTMN
AKLLMDPKRQIFLDQNMLAVIDELMQALNFNSETVPQKSS
LEEPDFAKTKIKLCILLHAFRIRAVTIDRVMSYLNAS
SEQ ID NO: 98:
human IL-12 P35 subunit mutein 3
(no signal peptide)
RNLPVATPDPGMFPCLHHSQNLLRAVSNMLQKARQTLEFY
PCTSEEIDHEDITKDKTSTVEACLPLELTKNESCLNSRET
SFITNGSCLASRKTSFMMALCLSSIYEDLKMYQVEFKTMN
AKLLMDPKRQIFLDQNMLAVIDELMQALNFNSETVPQKSS
LEEPDFYKTAIKLCILLHAFRIRAVTIDRVMSYLNAS
SEQ ID NO: 99:
human IL-12 P35 subunit mutein 4
(no signal peptide)
RNLPVATPDPGMFPCLHHSQNLLRAVSNMLQKARQTLEFY
PCTSEEIDHEDITKDKTSTVEACLPLELTKNESCLNSRET
SFITNGSCLASRKTSFMMALCLSSIYEDLKMYQVEFKTMN
AKLLMDPKRQIFLDQNMLAVIDELMQALNFNSETVPQKSS
LEEPDFHKTHIKLCILLHAFRIRAVTIDRVMSYLNAS

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skills in the art will be recognized, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the Background of the “Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend on both from the independent claim and from each of the prior dependent claims for each and every claim so long as the prior claim provides a proper antecedent basis for a claim term or element.

What is claimed is:

1. An activatable monomeric cytokine-scFc fusion protein comprising:
in a linear sequence from amino to carboxy terminus:
a P1-L-Fc-L-P2-L-Fc fusion protein; or
a P1-L-Fc-L-P2-L-Fc-L-P3 fusion protein;
wherein P1, P2, and P3 are each a biologically active moiety, or portion thereof;
wherein L is a non-cleavable linker or a cleavable linker that is cleaved by enzymes enriched in tumor tissues, and L is optional; and
wherein Fc is an antibody Fc fragment.

2. The activable fusion protein of claim 1, wherein at least one of:
the cleavable or non-cleavable linker is up to 50 amino acids in length;
the cleavable linker is cleaved by enzymes enriched in tumor tissues in vivo; or
cleavage of the cleavable linker by tumor-specific enzymes activates the activable fusion protein in a tumor microenvironment

3. The activable fusion protein of claim 1, wherein at least one of:
P1, P2, and P3 are different biologically active moieties, but at least one of them is a cytokine;
P1, P2, P3, Fc, and L are human sequences;
P1, P2, or P3, is a tumor-targeting antibody or an antigen-binding fragment, a cytokine receptor subunits or subunits that increase or reduce a cytokine activity of the fusion protein before cleavage of the linker, or a cytokine subunit that forms an intramolecular heterodimer; or
P1, P2, or P3 is a cytokine or a mutein that comprises one or more mutations with increased or reduced binding affinity to its cognate receptor, and a wild-type or mutant cytokine is used in the activable fusion protein.

4. The activable fusion protein of claim 1, wherein the antibody or antigen-binding fragment at least one of: specifically targets the fusion protein to tumor tissues, or activates residing effector cells for cancer treatment.

5. The activable fusion protein of claim 1, wherein the Fc fragments form a dimeric Fc region by inter-moiety disulfide bonds between cysteines in a hinge region that hold the dimeric Fc region is a functional unit to block the activity of any one of P1, P2, or P3, or wherein the Fc is a wild-type or a mutated Fc.

6. The activable fusion protein of claim 1, further comprising at least one of:
one or more protein domains at an amino-, a carboxy-, or both the amino- and carboxy-terminus of the fusion protein, wherein the one or more protein domains is an antibody, binding fragments thereof, Fc region, cytokine or receptor; or
the fusion protein comprises one or more split domains of an antibody, a cytokine, or a receptor linked with cleavable or non-cleavable linkers.

7. The activable fusion protein of claim 1, wherein the cleavable linker is cleaved by a protease selected from matrix metallopeptidase (MMP)-1, MMP2, MMP3, MMP7, MMP9, MMP 10, MMP 11, MMP 12, MMP 13, MMP 14, MMP 15, MMP 16, MMP 17, MMP 19, MMP20, MMP21, uPA, FAPa, or Cathepsin B; or the cleavable linker is cleaved by a caspase selected from Caspase 1, Caspase 2, Caspase 3, Caspase 4, Caspase 5, Caspase 6, Caspase 7, Caspase 8, Caspase 9, Caspase 10, Caspase 11 and Caspase 12.

8. The activable fusion protein of claim 1, wherein the at least one of:
The biologically active moiety is an antibody, or an antigen-binding fragment, a cytokine receptor, a cytokine, or combinations thereof, a tumor-targeting antibody binding portion;
an antigen is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), a Claudin family protein;
the biologically active moiety comprises an antibody binding portion specific to a T-cell marker selected from CTLA-4, PD-1, Lag3, S15, B7H3, B7H4, TCR-alpha, TCR-beta, or TIM-3;
the biologically active moiety comprises a target-binding portion specific to a T-cell activator selected from CD3, 41BB, or OX40;
the biologically active moiety comprises an antibody binding portion specific to an antigen-presenting cell marker selected from PD-L1, CD40, CD24, B7H3, TGF-beta receptor, TNFR family members 1 to 20, CD80, CD86, FLT3, CD11c, CD8-alpha, 5B6 (CLEC9A), CD1c, CD11 b, CD13, CD33, HLA-DR, CD141, CD1a, CD32, CD45, CD80, CD86, CD207, CD2, CD7, CD45RA, CD68, CD123, CD303, or CD304; or
at least one of the biologically active moieties comprises: IL-1, IL-2, IL-4, IL-5, IL-6, IL-7, IL-8, IL-10, IL-11, IL-12, IL-13, IL-15, IL-18, IL-21, interferons α, β, or γ, colony-stimulating factors (CSFs), granulocyte-macrophage CSF, tumor necrosis factor alpha, or tumor necrosis factor beta.

9. The activable fusion protein of claim 1, wherein a cancer is selected from the group consisting of: acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma; or the human autoimmune disease is selected from autoimmune diseases or disorders, inflammatory skin diseases, psoriasis, dermatitis, atopic dermatitis; responses associated with inflammatory bowel diseases, Crohn's disease, ulcerative colitis; dermatitis; allergic conditions, eczema, asthma; rheumatoid arthritis; systemic lupus erythematosus (SLE), lupus nephritis, cutaneous lupus; diabetes mellitus, type 1 diabetes mellitus, insulin-dependent diabetes mellitus; multiple sclerosis and juvenile-onset diabetes.

10. The activable fusion protein of claim 1, wherein the activable fusion protein has at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

11. The activable fusion protein of claim 1, made by a method comprising:
expressing a nucleic acid that encodes an activatable monomeric cytokine-scFc fusion protein comprising:
in a linear sequence from amino to carboxy terminus:
a P1-L-Fc-L-2P-L-Fc fusion protein; or
a P1-L-Fc-L-P2-L-Fc-L-P3 fusion protein;
wherein P1, P2, and P3 are each a biologically active moiety,
wherein L is a cleavable or non-cleavable linker that is cleaved by enzymes enriched in tumor tissues; and
wherein Fc is an antibody Fc fragment.

12. A method of treating a cancer or an autoimmune disease patient providing a subject in need thereof with an activatable monomeric cytokine-scFc fusion protein comprising:
providing the cancer or autoimmune disease patient with an effective amount of the activatable monomeric cytokine-scFc fusion protein sufficient to treat the cancer or the autoimmune disease comprising a linear sequence from amino to carboxy terminus:
a P1-L-Fc-L-2P-L-Fc fusion protein; or
a P1-L-Fc-L-P2-L-Fc-L-P3 fusion protein;
wherein P1, P2, and P3 are each a biologically active moiety, or portion thereof,
wherein L is a cleavable or non-cleavable linker that is cleaved by enzymes enriched in tumor tissues; and
wherein Fc is an antibody Fc fragment.

13. The method of claim 12, wherein at least one of:
the cleavable or non-cleavable linker is up to 50 amino acids in length;
the cleavable linker is cleaved by enzymes enriched in tumor tissues in vivo; or
cleavage of the cleavable linker by tumor-specific enzymes activates the activable fusion protein in a tumor microenvironment

14. The method of claim 12, wherein at least one of:
P1, P2, and P3 are different biologically active moieties, but at least one of them is a cytokine;
P1, P2, P3, Fc, and L are human sequences;
P1, P2, or P3, is a tumor-targeting antibody or an antigen-binding fragment, a cytokine receptor subunits or subunits that increase or reduce a cytokine activity of the fusion protein before cleavage of the linker, or a cytokine subunit that forms an intramolecular heterodimer; or
P1, P2, or P3 is a cytokine or a mutein that comprises one or more mutations with increased or reduced binding affinity to its cognate receptor, and a wild-type or mutant cytokine is used in the activable fusion protein.

15. The method of claim 12, wherein the antibody or antigen-binding fragment at least one of: specifically targets the fusion protein to tumor tissues, or activates residing effector cells for cancer treatment.

16. The method of claim 12, wherein the Fc fragments form a dimeric Fc region by inter-moiety disulfide bonds between cysteines in a hinge region that hold the dimeric Fc region is a functional unit to block the activity of any one of P1, P2, or P3, or wherein the Fc is a wild-type or a mutated Fc.

17. The method of claim 12, further comprising at least one of:
one or more protein domains at an amino-, a carboxy-, or both the amino- and carboxy-terminus of the fusion protein, wherein the one or more protein domains is an antibody, binding fragments thereof, Fc region, cytokine or receptor; or
the fusion protein comprises one or more split domains of an antibody, a cytokine, or a receptor linked with cleavable or non-cleavable linkers.

18. The method of claim 12, wherein the cleavable linker is cleaved by a protease selected from matrix metallopeptidase (MMP)-1, MMP2, MMP3, MMP7, MMP9, MMP10, MMP 11, MMP12, MMP13, MMP14, MMP15, MMP16, MMP17, MMP19, MMP20, MMP21, uPA, FAPa, or Cathepsin B; or the cleavable linker is cleaved by a caspase selected from Caspase 1, Caspase 2, Caspase 3, Caspase 4, Caspase 5, Caspase 6, Caspase 7, Caspase 8, Caspase 9, Caspase 10, Caspase 11 and Caspase 12.

19. The method of claim 12, wherein the at least one of:
The biologically active moiety is an antibody, or an antigen-binding fragment, a cytokine receptor, a cytokine, or combinations thereof, a tumor-targeting antibody binding portion;
an antigen is selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), a Claudin family protein;
the biologically active moiety comprises an antibody binding portion specific to a T-cell marker selected from CTLA-4, PD-1, Lag3, S15, B7H3, B7H4, TCR-alpha, TCR-beta, or TIM-3;
the biologically active moiety comprises a target-binding portion specific to a T-cell activator selected from CD3, 41BB, or OX40;
the biologically active moiety comprises an antibody binding portion specific to an antigen-presenting cell marker selected from PD-L1, CD40, CD24, B7H3, TGF-beta receptor, TNFR family members 1 to 20, CD80, CD86, FLT3, CD11c, CD8-alpha, 5B6 (CLEC9A), CD1c, CD11 b, CD13, CD33, HLA-DR, CD141, CD1a, CD32, CD45, CD80, CD86, CD207, CD2, CD7, CD45RA, CD68, CD123, CD303, or CD304; or
at least one of the biologically active moieties comprises: IL-1, IL-2, IL-4, IL-5, IL-6, IL-7, IL-8, IL-10, IL-11, IL-12, IL-13, IL-15, IL-18, IL-21, interferons α, β, or γ, colony-stimulating factors (CSFs), granulocyte-macrophage CSF, tumor necrosis factor alpha, or tumor necrosis factor beta.

20. The method of claim 12, wherein a cancer is selected from the group consisting of: acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma; or the human autoimmune disease is selected from autoimmune diseases or disorders, inflammatory skin diseases, psoriasis, dermatitis, atopic dermatitis; responses associated with inflammatory bowel diseases, Crohn's disease, ulcerative colitis; dermatitis; allergic conditions, eczema, asthma; rheumatoid arthritis; systemic lupus erythematosus (SLE), lupus nephritis, cutaneous lupus; diabetes mellitus, type 1 diabetes mellitus, insulin-dependent diabetes mellitus; multiple sclerosis and juvenile-onset diabetes.

21. The method of claim 12, wherein the activable fusion protein has at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

22. A nucleic acid that encodes an activatable monomeric cytokine-scFc fusion protein comprising:
in a linear sequence from amino to carboxy terminus:
a P1-L-Fc-L-2P-L-Fc fusion protein; or
a P1-L-Fc-L-P2-L-Fc-L-P3 fusion protein;
wherein P1, P2, and P3 are each a biologically active moiety,
wherein L is a cleavable or non-cleavable linker that is cleaved by enzymes enriched in tumor tissues; and
wherein Fc is an antibody Fc fragment.

23. The nucleic acid of claim 22, wherein the nucleic acid encodes an activable fusion protein having at least 90, 91, 92, 93, 94, 95, 96, 97, 98, 99, or 100 sequence identify with SEQ ID NOS: 60; 60+61; 62; 62+63; 64; 64+65; 66; 66+65; 67; 67+68; 69 to 87, 94, 95, 96, 97, 98, or 99.

24. A vector that expresses a nucleic acid that encodes an activatable monomeric cytokine-scFc fusion protein comprising:
in a linear sequence from amino to carboxy terminus:
a P1-L-Fc-L-2P-L-Fc fusion protein; or
a P1-L-Fc-L-P2-L-Fc-L-P3 fusion protein;
wherein P1, P2, and P3 are each a biologically active moiety,
wherein L is a cleavable or non-cleavable linker that is cleaved by enzymes enriched in tumor tissues; and
wherein Fc is an antibody Fc fragment.

25. A host cell that comprises a vector that expresses a nucleic acid that encodes an activatable monomeric cytokine-scFc fusion protein comprising:
in a linear sequence from amino to carboxy terminus:
a P1-L-Fc-L-2P-L-Fc fusion protein; or
a P1-L-Fc-L-P2-L-Fc-L-P3 fusion protein;
wherein P1, P2, and P3 are each a biologically active moiety,
wherein L is a cleavable or non-cleavable linker that is cleaved by enzymes enriched in tumor tissues; and
wherein Fc is an antibody Fc fragment.

26. An engineered human IL-12P35 subunit mutein that does not include a signal peptide, wherein residues that substitute residues 158 K, 162 E, 163 E, 165 D, 167 Y, 168 K, 170 K and 172 K, are selected from A, H, D, E, R and K.
